(19) United States
(12) Patent Application Publication
(10) Pub. No.: US 2006/0094651 A1
(43) Pub. Date: May 4, 2006

(54) Formulations and methods of production of FGF-20

(76) Inventors: Cyrus Karkaria, Branford, CT (US); Tracy Chen, Clinton, CT (US); Rajeev Chillakuru, Chester, CT (US); Gan Wei, Hamden, CT (US); Saravanamoorthy Rajendran, Branford, CT (US)

Correspondence Address: Jenell Lawson;Intellectual Property, CuraGen Corporation, 555 Long Wharf Drive, New Haven, CT, 06551, US

(21) Appl. No.: 10/980,370

(22) Filed: Nov. 3, 2004

(51) Int. Cl. A61K 38/18; A61K 31/724

(52) U.S. Cl. 514/12; 514/58

(57) ABSTRACT

The present invention provides improved formulations comprising FGF-20, its fragments, derivatives, variants, homologs, analogs, or a combination thereof, and improved methods for production.

### 1. FIELD OF THE INVENTION

The present invention relates to improved formulations comprising FGF-20, its fragments, derivatives, variants, homologs, analogs, or a combination thereof, and improved methods for production.

### 2. BACKGROUND OF THE INVENTION

The fibroblast growth factor (“FGF”) family consists of more than 20 members, each containing a conserved amino acid core (see, e.g., Powers et al., Endocr. Relat. Cancer, 7(3):65-197 (2000)). FGFs regulate diverse cellular functions such as growth, survival, apoptosis, motility, and differentiation (see, e.g., Szebenyi et al., Int. Rev. Cytol., 185:45-106 (1999)). Members of the FGF family are also involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7(3):165-197 (2000); and Szebenyi et al., Int. Rev. Cytol. 185:45-106(1999)).

Through a homology-based genomic mining process, a novel human FGF, FGF-20, was discovered. See U.S. patent application Ser. No. 09/494,585, filed Jan. 13, 2000, and Ser. No. 09/609,543, filed Jul. 3, 2000, the disclosure of each references is incorporated herein by reference. The amino acid sequence of FGF-20 shows close homology with human FGF-9 (70% identity) and FGF16 (64% identity).

Recombinant full length FGF-20 has been shown to induce a proliferative response in mesenchymal and epithelial cells, but not in human smooth muscle, erythroid, or endothelial cells (see, e.g., Jeffers et al., Cancer Res. 61(7):3131-3138(2001)). FGF-20 and its variants or derivatives have also been shown to be effective in preventing and/or treating certain diseases, such as oral mucositis (see U.S. patent application Ser. No. 10/435,087, filed May 9, 2003), inflammatory bowel disease (“IBD”) (see U.S. patent application Ser. No. 09/992,840, filed Nov. 6, 2001, Ser. No. 10/011,364, filed Nov. 16, 2001, and Ser. No. 10/321,962, filed Dec. 16, 2002), osteoarthritis (see U.S. patent application Ser. No. 10/842,206, filed May 10, 2004), and certain diseases related to central nerve system, such as Parkinson's Disease, and certain diseases related to cardiovascular system, such as stroke (see U.S. patent application Ser. No. 10/842,206, filed May 10, 2004). FGF-20 and its variants or derivatives have also been shown to be effective in preventing and/or treating symptoms associated with radiation exposure (see U.S. patent application Ser. No. 10/842,179, filed May 10, 2004). The disclosure of each reference is incorporated herein by reference in its entirety.

Therefore, there is a great need for pharmaceutical formulations comprising FGF-20 and/or its variants or derivatives that are suitable for clinical uses, of which formulations are stable and can be produced at a commercial scale.

Citation or discussion of a reference herein shall not be construed as an admission that such is prior art to the present invention.

### 3. SUMMARY OF THE INVENTION

The present invention provides improved formulations comprising a fibroblast growth factor, preferably FGF-20, or its fragments, derivatives, variants, homologs, analogs, or a combination thereof. The present invention also provides improved production methods for isolating one or more CG53135 proteins.

In one embodiment, the present invention provides a formulation comprising about 0.1-1 M arginine in a salt form, sulfobutyl ether Beta-cyclodextrin sodium, or sucrose, about 0.01-0.1 M sodium phosphate monobasic (NaH2PO4.H2O), about 0.01%-0.1% weight/volume (“w/v”) polysorbate 80 or polysorbate 20, and an isolated fibroblast growth factor (“FGF”). In a specific embodiment, the concentration of the FGF in the formulations of the invention is about 0.005 mg/ml to about 50 mg/ml. The FGF protein is preferably a CG53135 protein. In a specific embodiment, the formulations of the present invention comprise one or more isolated proteins selected from the group consisting of: (a) a protein comprising an amino acid sequence of SEQ ID NOs:2, 4, 7, 10, 22, 24, 26, 28, 30, 32, 34, 36, 38, or 40; (b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and (c) a fragment of the protein of (a) or (b), which fragment retains cell proliferation stimulatory activity. In some embodiments, the formulations of the invention comprise one or more isolated proteins, where the concentration of the proteins is of 0.5-30 mg/ml. In a specific embodiment, the concentration of the proteins is 10 mg/ml. In some embodiments, the formulations of the invention are lyophilized or spray dried.

In a specific embodiment, the formulations of the invention comprise an arginine in a salt form, which is selected from the group consisting of arginine, arginine sulfate, arginine phosphate, and arginine hydrochloride. In one embodiment, the arginine in a salt form, sulfobutyl ether Beta-cyclodextrin sodium, or sucrose in the formulations of the invention has a concentration of 0.01-0.7 M, preferably 0.5 M.

In another embodiment, the sodium phosphate monobasic in the formulations of the invention has a concentration of 0.05 M. In another embodiment, polysorbate 80 or polysorbate 20 of the formulations of the invention is 0.01% (w/v). In one embodiment, the formulations of the invention comprise polysorbate 80. In another embodiment, the formulations of the invention comprise polysorbate 20.

In a specific embodiment, a formulation of the invention comprises about 10 mg/ml of an isolated protein comprising an amino acid sequence of SEQ ID NO:24, 0.5 M arginine sulfate, 0.05 M sodium phosphate monobasic, and 0.01% (w/v) polysorbate 80. In another specific embodiment, a formulation of the invention comprises about 10 mg/ml of an isolated protein comprising an amino acid sequence of SEQ ID NO:2, 0.5 M arginine sulfate, 0.05 M sodium phosphate monobasic, and 0.01% (w/v) polysorbate 80. In another embodiment, a formulation of the invention comprises 0.5 M arginine sulfate, 0.05 M sodium phosphate monobasic, 0.01% (w/v) polysorbate 80, and about 10 mg/ml of a mixture of isolated proteins, wherein said proteins comprise a first protein comprising an amino acid sequence of SEQ ID NO:24, and a second protein comprising an amino acid sequence of SEQ ID NO:2. In a specific embodiment, a formulation of the invention further comprises one or more isolated proteins, wherein said proteins comprise an amino acid sequence selected from the group consisting of SEQ ID NOs:26, 28, 30 and 32. In some embodiments, the formulations of the invention comprise one or more isolated proteins that are carbamylated.

In another embodiment, the present invention provides methods of increasing solubility of a fibroblast growth factor (“FGF”) in an aqueous solution by adding arginine in a salt form, sulfobutyl ether Beta-cyclodextrin sodium, or sucrose, or a combination thereof to said solution to a final concentration of 0.01-1 M. In some embodiment, the fibroblast growth factor is an isolated CG53135 protein. In a specific embodiment, the fibroblast growth factor is an isolated protein comprising an amino acid sequence selected from the group consisting of SEQ ID NOs:2, 4, 7, 10, 22, 24, 26, 28, 30, 32, 34, 36, 38, and 40.

In some embodiments, an arginine in a salt form is selected from the group consisting of arginine, arginine sulfate, arginine phosphate, and arginine hydrochloride. In one embodiment, the final concentration of arginine in a salt form is 0.01-0.7 M, preferably 0.5 M. In some embodiments, the methods of the invention further comprise adding acetate, succinate, tartrate, or a combination thereof to the solution to increase the solubility of the protein. Preferably, the acetate, succinate, tartrate, or a combination thereof has a final concentration of 0.01-0.2 M in the solution.

In another embodiment, the present invention provides a method of producing an isolated protein comprising the steps of: (1) fermenting an E. coli cell containing a vector comprising SEQ ID NO:8; (2) chilling the fermented culture to 10-15° C.; (3) diluting the chilled culture with a lysis buffer comprising 50-100 mM sodium phosphate, 60 mM ethylene diamine tetraacetic acid, 7.5 mM DTT, and 3.5-5 M urea; (4) lysing the cells in the diluted culture; (5) loading the resultant cell lysate onto a pre-equilibrated cation exchange column, and flushing the column with a buffer comprising 50-100 mM sodium phosphate, 40 mM EDTA, 10 mM sodium sulfate, and 3-5 M urea; (6) washing the flushed column with a buffer comprising 50-100 mM sodium phosphate, 5 mM EDTA, 10-25 mM sodium sulfate, and 2.22 mM dextrose; (7) washing the column again with an elution buffer comprising 50-100 mM sodium phosphate, 5 mM EDTA, 150-250 mM sodium sulfate, and 0.5-1 M L-arginine; (8) loading the resultant eluate onto a hydrophobic interaction chromatography column pre-equilibrated with 50-100 mM sodium phosphate, 150-250 mM sodium sulfate, 5 mM EDTA, and 1 M arginine; (9) washing the resulting column with a solution comprising 100-250 mM sodium phosphate, 5 mM EDTA, and 0.8-1 M arginine; and (10) washing the column again with a solution comprising 50-100 mM sodium phosphate, 5 mM EDTA, and 0.1-0.3 M arginine to elute the protein. In a specific embodiment, the method further comprises the steps of: (11) concentrating the resultant eluate; (12) filtering the retentate obtained together with a solution comprising 50 mM sodium phosphate, 0.5 M arginine; (13) concentrating the filtered retentate; and (14) filtering the concentrated retentate.

In some embodiments, the cells are fermented by a method comprising the steps of: (a) culturing E. coli cells containing a vector comprising SEQ ID NO:8 to exponential growth phase with 2.5 to 4.5 OD600 units in a chemically defined seed medium; (b) inoculating cells of step (a) to a seed medium and culturing the cells to an exponential growth phase with 3.0 to 5.0 OD600 units; (c) transferring the cells of step (b) to a chemically defined batch medium; (d) culturing the cells of step (c) to 25-35 units OD600, and adding additional chemically defined medium with a feeding rate of 0.7 g/kg broth/minute; (e) culturing the cells of step (d) to 135 to 165 units OD600; and (f) culturing the cells of step (e) for about four hours.

The present invention also provides isolated proteins produced by the methods of the invention.

Uses of the formulations of the invention for preventing and/or treating a disease, e.g., alimentary mucositis, arthritis, a disorder or symptom associated with radiation exposure, a disorder of central nerve system or cardiovascular system, are also provided.

3.1 Terminology

As used herein, the term “about” in the context of a given numerate value or range refers to a value or range that is within 20%, preferably within 10%, and more preferably within 5% of the given value or range.

As used herein, the term “CG53135”, refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20 (see U.S. patent application Ser. No. 09/494,585); CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20); CG53135-12 (SEQ ID NOs:21 and 22) represent a single nucleotide polymorphism (“SNP”) of FGF-20 where one amino acid in CG53135-12 is different from SEQ ID NO:2 (the aspartic acid at position 206 is changed to asparagine, “²⁰⁶D? N”). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1 shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic acid molecules that can hybridize to FGF-20 under stringent hybridization conditions.

TABLE 1
Summary of some of the CG53135 family members

| | SEQ ID NO | |
|---|---|---|
| Name | (DNA/Protein) | Brief Description |
| CG53135-01 | 1 and 2 | FGF-20 wild type, stop codon removed |
| CG53135-02 | 3 and 4 | Codon optimized, amino acids 2-54 (as numbered in SEQ ID |
| | | NO: 2) were removed |
| CG53135-03 | 5 and 2 | FGF-20 wild type |
| CG53135-04 | 6 and 7 | Amino acids 20-51 (as numbered in SEQ ID NO: 2) were removed, |
| | | also valine at position 85 is changed to alanine (“⁸⁵V? A”) |
| CG53135-05 | 8 and 2 | Codon optimized, full length FGF-20 |
| CG53135-06 | 9 and 10 | Amino acids 20-51 (as numbered in SEQ ID NO: 2) were removed |
| CG53135-07 | 11 and 12 | Protein consisting of amino acids 1-18 (as numbered in SEQ ID NO: 2) |
| CG53135-08 | 13 and 14 | Protein consisting of amino acids 32-52 (as numbered in SEQ ID NO: 2) |
| CG53135-09 | 15 and 16 | Protein consisting of amino acids 173-183 (as numbered in SEQ ID NO: 2) |
| CG53135-10 | 17 and 18 | Protein consisting of amino acids 192-211 (as numbered in SEQ ID NO: 2) |
| CG53135-11 | 19 and 20 | Protein consisting of amino acids 121-137 (as numbered in SEQ ID NO: 2) |
| CG53135-12 | 21 and 22 | FGF-20 SNP, aspartic acid at position 206 is changed to |
| | | asparagines (“²⁰⁶D? N”) as compared to CG53135-01 |
| CG53135-13 | 23 and 24 | CG53135-05 minus first 2 amino acids at the N-terminus |
| CG53135-14 | 25 and 26 | CG53135-05 minus first 8 amino acids at the N-terminus |
| CG53135-15 | 27 and 28 | CG53135-05 minus first 11 amino acids at the N-terminus |
| CG53135-16 | 29 and 30 | CG53135-05 minus first 14 amino acids at the N-terminus |
| CG53135-17 | 31 and 32 | CG53135-05 minus first 23 amino acids at the N-terminus |
| IFC 250059629 | 33 and 34 | In frame clone, open reading frame comprising a nucleotide |
| | | sequence encoding amino acids 63-196 of FGF-20 (SEQ ID NO: 2) |
| IFC 250059669 | 35 and 36 | In frame clone, open reading frame comprising a nucleotide |
| | | sequence encoding amino acids 63-211 of FGF-20 (SEQ ID NO: 2) |
| IFC 317459553 | 37 and 38 | In frame clone, open reading frame comprising a nucleotide |
| | | sequence encoding amino acids 63-194 of FGF-20 (SEQ ID NO: 2) |
| | | with ¹⁵⁹G? E |
| IFC 317459571 | 39 and 40 | In frame clone, open reading frame comprising a nucleotide |
| | | sequence encoding amino acids 63-194 of FGF-20 (SEQ ID NO: 2) |
| IFC 250059596 | 41 and 10 | In frame clone, open reading frame comprising a nucleotide |
| | | sequence encoding amino acids 1-19 and 52-211 of FGF-20 (SEQ |
| | | ID NO: 2) |
| IFC 316351224 | 41 and 10 | In frame clone, open reading frame comprising a nucleotide |
| | | sequence encoding amino acids 1-19 and 52-211 of FGF-20 (SEQ |
| | | ID NO: 2). |

As used herein, the term “effective amount” refers to the amount of a therapy (e.g., a formulation comprising a CG53135 protein) which is sufficient to reduce and/or ameliorate the severity and/or duration of a disease (e.g., alimentary mucositis) or one or more symptoms thereof, prevent the advancement of a disease, cause regression of a disease, prevent the recurrence, development, or onset of one or more symptoms associated with a disease, or enhance or improve the prophylactic or therapeutic effect(s) of another therapy.

As used herein, the term “FGF-20” refers to a protein comprising an amino acid sequence of SEQ ID NO:2, or a nucleic acid sequence encoding such a protein or the complementary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2× sodium chloride/sodium citrate (SSC), 0.01% bovine serum albumin (BSA). In another non-limiting example, stringent hybridization conditions are hybridization at 6×SSC at about 45° C., followed by one or more washes in 0.1×SSC, 0.2% sodium dodecyl sulfate (SDS) at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “stability” in the context of a protein formulation, refers to the ability of a particular protein formulation to maintain the native, active structure of a protein as the protein is exposed to thermo-mechanical stresses over time. In some embodiments, stability of a protein formulation generally refers to the tendency of a protein formulation to form biologically inactive and/or insoluble aggregates of the protein as a result of exposure of the protein to thermo-mechanical stresses, as well as the tendency of a protein formulation to form biologically inactive and/or insoluble aggregates of the protein as a result of interaction with interfaces and surfaces that are destabilizing, such as hydrophobic surfaces and interfaces. A related parameter to the “stability” of a protein formulation is its solubility in that higher molecular weight aggregates and denatured forms of a protein, including partially denatured forms of a protein, which are generally less soluble than their non-aggregated, lower molecular weight counterparts and native forms of the protein. Another related parameter to the “stability” of a protein formulation is the protein concentration in that physically stable formulations may become less physically stable as the concentration of the protein is increased or decreased.

### 4. BRIEF DESCRIPTION OF THE DRAWINGS

FIG. 1 shows an example of manufacturing a drug product comprising one or more CG53135 proteins.

FIG. 2 (A) shows SDS-PAGE analysis (gel code blue) of CG53135 produced by Process 1 and Process 2 (as described in Section 6), respectively. Lane 1: molecular weight markers (kDa); lane 24: purified CG53135 (10 μg), reduced. Lane 5-8: Process 1 reference standard DEV10 (720, 380, 45, and 28 ng) reduced. (B) shows SDS-PAGE analysis (silver stain) of CG53135. Lane 1: molecular weight markers (kDa) are shown on the left; lane 3: purified CG53135 by Process 2 (5 μg); lane 5: purified CG53135 reference standard Process 1 (5 μg); lane 7: purified CG53135 Process 2 (10 μg); lane 9: purified CG53135 reference standard Process 1 (10 μg).

FIG. 3 shows RP-HPLC analysis of CG53135 purified by Process 1 and Process 2, respectively (Process 1 is represented by the solid line).

FIG. 4 shows SEC-HPLC analysis of CG53135 purified by Process 1 and Process 2, respectively.

FIG. 5 shows host cell protein analysis of CG53135 E. coli purified product (by Process 1 and Process 2, respectively).

FIG. 6 shows Western Blot analysis of CG53135 purified by Process 1 and Process 2, respectively. Western blot was probed with anti-CG53135-05 antibody. Lane 1: molecular weight marker (kDa); lane 3: CG53135 purified by Process 2 (10 μg); lane 5: CG53135 purified by Process 1 (10 μg).

FIG. 7 shows RP-HPLC identification analysis of CG53135 purified by Process 1 and Process 2, respectively. Process 2 is represented by the dashed line.

FIG. 8 shows tryptic map of CG53135 purified by Process 1 and Process 2, respectively.

FIG. 9 shows circular dichroism spectroscopy analysis of CG53135 produced by Process 1 and Process 2, respectively. The lower (grey) trace represents Process 1, and the upper (black) trace represents Process 2.

FIG. 10 shows near UV circular dichroism spectroscopy analysis of CG53135 purified by Process 1 and Process 2, respectively. The upper (grey) trace is the near UV CD spectrum of Process 1 and the black trace (lower) is the near UV CD spectrum of Process 2.

FIG. 11 shows second derivative absorbance spectra for Process 1 (grey trace) and Process 2 (black trace).

FIG. 12 shows temperature melting curves for Process 1 and Process 2, respectively, by differential scanning calorimetry.

### 5. DETAILED DESCRIPTION OF THE INVENTION

The present invention provides improved formulations comprising one or more CG53135 proteins, which are more stable and soluble, and can be easily lyophilized by commercial equipments. The improved formulations comprise 0.01-1 M of a stabilizer, such as arginine in a salt form, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more isolated CG53135 proteins. In a specific embodiment, the concentration of CG53135 protein(s) in the improved formulations of the invention is less than 50 mg/ml, less than 30 mg/ml, less than 10 mg/ml, less than 5 mg/ml, or less than 1 mg/ml. In another embodiment, the concentration of CG53135 protein(s) in the improved formulations of the invention is between 0.005-50 mg/ml. In a preferred embodiment, the formulation is lyophilized.

The present invention also provides methods for increasing solubility of a FGF protein in a solution (e.g., an aqueous solution). In one embodiment, the present invention provides a method for increasing solubility of a FGF protein in a solution by adding arginine in a salt form, sulfobutyl ether Beta-cyclodextrin sodium, or sucrose to the solution. In another embodiment, the present invention provides a method for increasing solubility or stability of a FGF protein in a solution by adding buffering salts such as acetate, succinate, tartrate, phosphate, or a combination thereof to the solution. In yet another embodiment, buffering salts such as acetate, succinate, tartrate, phosphate, or a combination thereof is added in combination with arginine in a salt form, sulfobutyl ether Beta-cyclodextrin sodium, or sucrose to the solution to increase the solubility of a FGF protein. The arginine in a salt form can be, but is not limited to, arginine, arginine sulfate, arginine phosphate, and arginine hydrochloride. In a preferred embodiment, arginine sulfate is used. In some embodiments, the final concentration of the arginine in a salt form, sulfobutyl ether Beta-cyclodextrin sodium, or sucrose is between 0.01 M to 1 M. In one embodiment, the final concentration of the arginine in a salt form, sulfobutyl ether Beta-cyclodextrin sodium, or sucrose is 0.5 M. In some embodiment, the final concentration of the buffering salts such as acetate, succinate, tartrate, phosphate, or a combination thereof is 0.05 M. In a preferred embodiment, the FGF protein is a FGF-20 protein, a fragment, a derivative, a variant, a homolog, or an analog of FGF-20, or a combination thereof.

The present invention further provides improved production methods for CG53135 proteins and/or formulations comprising one or more CG53135 proteins. The improved production methods allow for commercial scale production of CG53135 proteins and/or formulations comprising one or more CG53135 proteins. The improved production methods also allow for purifying CG53135 proteins to a high degree of purity. In some embodiments, the purity of the CG53135 purified by the improved production methods is at least 97%, at least 98%, at least 99%. In a preferred embodiment, the purity of CG53135 purified by the improved production methods is from 99% up to 100% (including 100%).

For clarity of disclosure, and not by way of limitation, the detailed description of the invention is divided into the following subsections:
(i) CG53135
(ii) Methods of Preparing CG53135
(iii) Characterization of CG53135
(iv) Pharmaceutical Compositions and Formulations

5.1 CG53135

The present invention provides for improved formulations comprising one or more CG53135 proteins and improved production methods. As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids, “FGF-20”), or its fragments, derivatives, variants, homologs, or analogs.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. In one embodiment, a CG53135 is CG53135-12 (SEQ ID NOs:21 and 22), which is a single nucleotide polymorphism (“SNP”) of FGF-20 (i.e., ²⁰⁶D? N). (For more detailed description of CG53135-12, see e.g., U.S. patent application Ser. No. 10/702,126, filed Nov. 4, 2003, the disclosure of which is incorporated herein by reference in its entirety.) Other examples of SNPs of FGF-20 are also described in U.S. patent application Ser. No. 10/435,087, the content of which is incorporated herein by reference.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

In another embodiment, CG53135 refers to a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein fragment includes, but is not limited to, residues 2-211, 3-211, 9-211, 12-211, 15-211, 24-211, 54-211, or 55-21 lof FGF-20 (SEQ ID NO:2). In one embodiment, CG53135 refers to a nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 which are altered by the substitution of different codons that encode a functionally equivalent amino acid residue within the sequence, thus producing a silent change. In a preferred embodiment, a wild-type FGF-20 nucleic acid sequence is codon optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations which result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2— groups, free COOH— groups, OH— groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, a-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Ca-methyl amino acids, and Na-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has improved solubility and/or an extended half-life in vivo using any methods known in the art. For example, Fc fragment of human IgG, or inert polymer molecules such as high molecular weight polyethyleneglycol (PEG) can be attached to a CG53135 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

Examples of prophylactic and/or therapeutic uses of CG53135 have been described in previously filed patent applications (see e.g., U.S. patent application Ser. Nos. 09/992,840, 10/011,364, 10/321,962, 10/435,087, 10/842,206, 10/842,179, and U.S. Pat. No. 6,797,695). The disclosure of each reference is incorporated by reference herein in its entirety.

5.2 Methods of Preparing CG53135

The present invention provides for formulations comprising one or more isolated CG53135 proteins and improved methods of production. In accordance with the methods described herein, CG53135 proteins employed in a formulation of the invention or produced by the production methods of the invention can have a purity in the range of 80 to 100 percent, or at least 80%, at least 85%, at least 90%, at least 95%, or at least 98%. In one embodiment, one or more CG53135 proteins employed in a formulation of the invention or produced by the production methods of the invention have a purity of at least 99%. In another embodiment, CG53135 is purified to apparent homogeneity, as assayed, e.g., by sodium dodecyl sulfate polyacrylamide gel electrophoresis. Any techniques known in the art can be used in purifying a CG53135 protein, including but are not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays as described in Section 5.3, infra. The purity of CG53135 can be assayed by any methods known in the art, such as but not limited to, gel electrophoresis. See Scopes, supra.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions which enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalovirus for effective expression of a CG53135 sequence (Foecking et al., 1986, Gene 45:101; and Cockett et al., 1990, Bio/Technology 8:2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors which direct the expression of high levels of fusion protein products that are readily purified may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific protease like enterokinase allows to cleave off the CG53135 protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, HeLa, COS, MDCK, 293, 3T3, W138, BT483, Hs578T, HTB2, BT20 and T47D, NSO (a murine myeloma cell line that does not endogenously produce any immunoglobulin chains), CRL7030 and HsS78Bst cells. Expression in a bacterial or yeast system can be used if post-translational modifications turn to be non-essential for a desired activity of CG53135. In a preferred embodiment, E. coli is used to express a CG53135 sequence.

For long term, high yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):I55-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-a-protected amino acids having protected side chains are added stepwise to a growing polypeptide chain linked by its C-terminal and to an insoluble polymeric support, i.e., polystyrene beads. The proteins are synthesized by linking an amino group of an N-a-deprotected amino acid to an a-carboxyl group of an N-a-protected amino acid that has been activated by reacting it with a reagent such as dicyclohexylcarbodiimide. The attachment of a free amino group to the activated carboxyl leads to peptide bond formation. The most commonly used N-a-protecting groups include Boc which is acid labile and Fmoc which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 is accomplished using conventional procedures, such as preparative HPLC using gel permeation, partition and/or ion exchange chromatography. The choice of appropriate matrices and buffers are well known in the art and so are not described in detail herein.

5.2.1 Improved Production Methods

The present invention provides improved manufacturing processes for producing compositions comprising one or more CG53135 proteins. The improved manufacturing processes provide benefits such as more stable and more pure drug product, and are also suitable for commercial scale production of a composition comprising one or more CG53135 proteins.

The present invention provides methods of isolating a protein, where the methods comprises the steps of: (1) fermenting a host cell, such as E. coli, that containing a vector, where the vector comprises a nucleotide sequence encoding a CG53135 protein. In a preferred embodiment, the vector comprises a codon-optimized, full length CG53135-05 (SEQ ID NO:8); (2) lysing the cultured cells. Cells may be lysed by any methods known in the art. In one embodiment, cells are lysed by homogenization. In another embodiment, the fermented cultured cells are chilled, and diluted with cell lysis buffer comprising 50-100 mM sodium phosphate, 60 mM EDTA, 7.5 mM DTT, 3.5-5 M urea, pH 7.2, and then lysed by, e.g., homogenization. In a preferred embodiment, polyethyleneimine (“PEI”) is added to the fermentation broth before homogenization; (3) purification by a cation exchange column. In a preferred embodiment, a pre-equilibrated expanded bed cation exchanger, such as STREAMLINE SP™ is used. In one embodiment, after the cation exchange column is loaded with the protein to be isolated, the column is flushed with additional equilibration buffer comprising 50-100 mM sodium phosphate, 40 mM EDTA, 10 mM sodium sulfate, 3-5 M urea, pH 7.0. The column may be further washed with a buffer comprising 50-100 mM sodium phosphate, 5 mM EDTA, 10-25 mM sodium sulfate, 2.22 M dextrose, pH 7.0. The elution buffer to elute the protein from the cation exchange column comprises, e.g., 50-100 mM sodium phosphate, 5 mM EDTA, 150-250 mM sodium sulfate, 0.5-1 M L-arginine, pH 7.0; and (4) further purification by using a hydrophobic interaction chromatography column (e.g., PPG 650M). In one embodiment, the hydrophobic interaction chromatography column, e.g., PPG 650M, is equilibrated and washed with 50-100 mM sodium phosphate, 150-250 mM sodium sulfate, 5 mM EDTA, 1 M arginine, pH 7.0. In another embodiment, the column is further washed with 100-250 mM sodium phosphate, 5 mM EDTA, 0.8-1 M arginine, pH 7.0. In another embodiment, the protein is eluted with 50-100 mM sodium phosphate, 5 mM EDTA, and 0.1-0.3 M arginine, pH 7.0.

In a preferred embodiment, the eluted protein from step (4) described above may be further purified by either one or both of the following steps: (5) further purification by filtering the eluted protein. In a preferred embodiment, a charged endotoxin binding filter (e.g., CUNO™ 30 ZA depth filter) is used. In one embodiment, the filter is first flushed with water for injection, and then with 50-100 mM sodium phosphate, 5 mM EDTA, 0.1-03 M arginine, pH 7.0; and (6) further purification by using a hydrophobic interaction chromatography column (e.g., Phenyl Sepharose HP Chromatography). In one embodiment, the column is equilibrated and washed with 50-100 mM sodium phosphate, 10-100 mM ammonium sulfate, 800-1000 mM sodium chloride, 0.5-1M arginine, pH 7.0. In another embodiment, the protein is eluted with 50-100 mM sodium phosphate, 0.5-1M arginine, pH 7.0.

A protein isolated by the methods of the present invention may be further concentrated and filtered to produce a drug product. Pharmaceutical carriers may be added to produce a desired formulation, such as formulations provided by the present invention.

5.3 Characterization of CG53135

The characteristics of the protein(s) purified by the production methods of the instant invention or the immediate products of the production methods of the instant invention (e.g., purity, various characters including the biological activity of CG53135) may be determined by methods known in the art. Compositions for use in therapy can be tested in suitable animal model systems prior to testing in humans, including but not limited to in rats, mice, chicken, cows, monkeys, rabbits, etc (examples of such tests can be found in U.S. patent application Ser. Nos. 09/992,840, 10/011,364, 10/321,962, 10/435,087, 10/842,206, and 10/842,179, the disclosure of each is incorporated herein by reference).

For examples, methods known in the art, such as but not limited to, sodium dodecyl sulphate polyacrylamide gel electrophoresis (“SDS-PAGE”), reversed phase high-performance liquid chromatography (“RP-HPLC”), size exclusion high-performance liquid chromatography (“SEC-HPLC”), Western Blot (e.g., host cell protein Western Blot), can be used to analyze the purity of the product of the manufacturing processes of the instant invention. In a preferred embodiment, a product of the manufacturing processes of the instant invention is at least 97%, at least 98%, or at least 99% pure by densitometry. In another preferred embodiment, a product of the manufacturing processes of the instant invention is more than 97%, more than 98%, or more than 99% pure by densitometry.

Methods known in the art, such as but not limited to, Western Blot, sequencing (e.g., N-terminal Edman sequencing), liquid chromatography (e.g., HPLC, RP-HPLC with both UV and electrospray mass spectrometric detection), mass spectrometry, total amino acid analysis, peptide mapping, SDS-PAGE, can be used to determine the identity of the product of the manufacturing processes of the instant invention. The secondary, tertiary and/or quaternary structure of a product of the manufacturing processes of the instant invention can analyzed by any method know in the art, for example, far UV circular dichroism spectrum can be used to analyze the secondary structure, near UV circular dichroism spectroscopy and second derivative UV absorbance spectroscopy can be used to analyze the tertiary structure, and light scattering SEC-HPLC can be used to analyze quaternary structure.

Potency of a product of the manufacturing processes of the instant invention can be measured by methods known in the art or any bioassays that measuring one or more biological activities of a CG53135 protein. In one embodiment, potency of a product of the manufacturing processes of the instant invention is measured by the ability of the product to stimulate cell growth of NIH 3T3 cells (for an example of such assay, see Section 6.5).

Other characters of a product of the manufacturing processes of the instant invention, such as safety (e.g., residual DNA, endotoxin, bioburden), pH, osmolarity, sulfyhydryl content, can also be analyzed by any method known in the art (for examples of some of these methods, see Section 6). Such methods are well known in the art and are not described in detail herein.

In one embodiment, a CG53135 protein reference standard, which is representative of the bulk drug substance from the improved manufacturing process, is prepared and characterized. Preferably, this reference standard is characterized for its purity, identity (e.g., molecular weight, amino acid sequence), potency, structure (e.g., secondary, tertiary and quaternary structures), safety (e.g., endotoxin, bioburden), and other characters, such as but not limited to, pH, osmolality, and sulfyhydryl content. Upon visual inspection, a CG53245 protein reference standard should be clear and colorless. Once a CG53135 protein reference is established, it can be used for, e.g., quality control of the manufacturing process, and as a positive control for other assays related to CG53135.

5.4 Pharmaceutica Compositions and Formulations

The present invention encompasses pharmaceutical compositions (including formulations) comprising one or more CG53135 proteins. The pharmaceutical compositions can be administered to a subject at a prophylactically or therapeutically effective amount to prevent and/or treat one or more diseases, such as inflammatory bowel disease (“IBD”), alimentary mucositis (including oral mucositis), arthritis, diseases associated with the central nerve system or cardiovascular system, and symptoms associated with radiation exposure. Various delivery systems are known and can be used to administer a composition comprising one or more CG53135 proteins. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, rectal, and oral routes. The compositions of the invention may be administered by any convenient route, for example, by infusion or bolus injection, by absorption through epithelial or mucocutaneous linings (e.g., oral mucosa, virginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, topical application, by injection, by infusion pump, by means of a suppository, or by means of an implant (the implant being of a reservoir with a porous, non-porous, or gelatinous material, including membranes, such as sialastic membranes, or fibers).

In some embodiments, a CG53135 nucleic acid can be administered in vivo to promote expression of their encoded proteins, by constructing the nucleic acid as part of an appropriate nucleic acid expression vector and administering it so that it becomes intracellular, e.g., by use of a retroviral vector, or by direct intramuscular or intradermal injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a CG53135 nucleic acid can be introduced intracellularly and incorporated within host cell DNA for expression, by homologous recombination.

The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in a salt form, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of welting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, micelles, emulsions, sphingomyelins, lipid-protein or lipid peptide complexes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulbse); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiments, the dosage of a composition comprising one or more G53135 proteins for administration in a human patient provided by the present invention is at least 0.001 mg/kg, at least 0.01 mg/kg, at least 0.1 mg/kg, at least 0.5 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 60 mg/kg, at least 70 mg/kg, at least 80 mg/kg, at least 90 mg/kg, at least 100 mg/kg, at least 150 mg/kg, or at least 200 mg/kg (as measured by Bradford assay). In another embodiment, the dosage of a composition comprising one or more CG53135 proteins for administration in a human patient provided by the present invention is between 0.001-300 mg/kg, between 0.01-300 mg/kg, between 0.1-300 mg/kg, between 0.5-250 mg/kg, between 1-200 mg/kg, between 1-150 mg/kg, between 1-125 mg/kg, between 1-100 mg/kg, between 1-90 mg/kg, between 1-80 mg/kg, between 1-70 mg/kg, between 1-60 mg/kg, between 1-50 mg/kg, between 140 mg/kg, between 1-35 mg/kg, between 1-30 mg/kg, between 1-25 mg/kg, between 1-20 mg/kg, between 1-15 mg/kg, between 1-10 mg/kg, or between 1-5 mg/kg (as measured by Bradford assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.1-300 mg/kg measured by Bradford assay, then the dosage is 0.033-100 mg/kg as measured by a UV assay.

In one embodiment, prior to administering the first full dose, each patient preferably receives a bolus injection of a small amount (e.g., 1/100 to 1/10 of the prescribed dose) of a composition of the invention to detect any acute intolerance. The injection site is examined one and two hours after the test. If no reaction is detected, then the full dose is administered.

The invention also provides kits for carrying out the therapeutic regimens of the invention. Such kits comprise in one or more containers prophylactically or therapeutically effective amounts of the composition of the invention in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe (single or dual chambered), preferably packaged in sterile form, for injecting the formulation, and/or a packaged alcohol pad. In a specific embodiment, a kit of the invention comprises pre-filled needles or syringes (single or dual chambered) that are pre-filled with a composition comprising one or more CG53135 proteins. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention sufficient for a single administration.

5.4.1 Improved Formulations and Methods to Increase Solubility of a FGF Protein

The present invention provides improved formulations comprising one or more FGFs, preferably one or more CG53135 proteins, and methods for increasing solubility of FGF proteins. The improved formulations are more stable and more favorable for commercial scale productions.

While not limited by any theory, the improved formulations are based partially on the discovery that high concentrations of arginine in a salt form, sulfobutyl ether Beta-cyclodextrin sodium, sucrose, acetate, succinate, or tartrate or a combination thereof can increase solubility of a growth factor, including FGF proteins. Accordingly, in one embodiment, the present invention provides a method of increasing solubility of a FGF protein in a solution (e.g., an aqueous solution) by adding arginine in a salt form, sulfobutyl ether Beta-cyclodextrin sodium, or sucrose to the solution. In another embodiment, the present invention provides a method for increasing solubility of a FGF protein in a solution by adding acetate, succinate, tartrate, or a combination thereof to the solution. In yet another embodiment, acetate, succinate, tartrate or a combination thereof is added in combination with arginine in a salt form, sulfobutyl ether Beta-cyclodextrin sodium, or sucrose to the solution to increase the solubility of a FGF protein. The arginine in a salt form can be, but is not limited to, arginine, arginine sulfate, arginine phosphate, and arginine hydrochloride. In a preferred embodiment, arginine sulfate is used. In some embodiments, the final concentration of the arginine in a salt form, sulfobutyl ether Beta-cyclodextrin sodium, or sucrose is between 0.01 M to 1 M. In one embodiment, the final concentration of the arginine in a salt form is 0.5 M. In some embodiment, the final concentration of the acetate, succinate, tartrate or a combination thereof is 0.01 to 0.2 M. In a preferred embodiment, the FGF protein in the formulation is a FGF-20 protein, a fragment, a derivative, a variant, a homolog, or an analog of FGF-20, or a combination thereof. In some embodiments, the FGF protein in the formulation is CG53135-01 (SEQ ID NO:2), CG53135-02 (SEQ ID NO: 4), CG53135-03 (SEQ ID NO:2), CG53135-04 (SEQ ID NO:7), CG53135-05 (SEQ ID NO: 2), CG53135-06 (SEQ ID NO: 10), CG53135-07 (SEQ ID NO:12), CG53135-08 (SEQ ID NO:14), CG53135-09 (SEQ ID NO:16), CG53135-10 (SEQ ID NO:18), CG53135-11 (SEQ ID NO:20), CG53135-12 (SEQ ID NO:22), CG53135-13 (SEQ ID NO:24), CG53135-14 (SEQ ID NO:26), CG53135-15 (SEQ ID NO:28), CG53135-16 (SEQ ID NO:30), CG53135-17 (SEQ ID NO:32), IFC 250059629 (SEQ ID NO:34), IFC 20059669 (SEQ ID NO:36), IFC 317459553 (SEQ ID NO:38), IFC 317459571 (SEQ ID NO:40), IFC 250059596 (SEQ ID NO:10), or IFC316351224 (SEQ ID NO:10), or any two or more combinations of CG53135 proteins. In one embodiment, the FGF proteins in the formulation comprise (1) a protein comprising an amino acid sequence of SEQ ID NO:2, and (2) a protein comprising an amino acid sequence of SEQ ID NO:24. In another embodiment, the FGF proteins in the formulation comprise (1) a protein comprising an amino acid sequence of SEQ ID NO:2, (2) a protein comprising an amino acid sequence of SEQ ID NO:24, (3) a protein comprising an amino acid sequence of SEQ ID NO:26, (4) a protein comprising an amino acid sequence of SEQ ID NO:28, (5) a protein comprising an amino acid sequence of SEQ ID NO:30, and (6) a protein comprising an amino acid sequence of SEQ ID NO:32. In another embodiment, the FGF proteins in the formulation comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, and (5) a protein comprising an amino acid sequence of SEQ ID NO:32. In another embodiment, a formulation of the invention comprises (1) a protein comprising an amino acid sequence of SEQ ID NO:32; (2) a protein comprising an amino acid sequence of SEQ ID NO:30, (3) a protein comprising an amino acid sequence of SEQ ID NO:28; and (4) a protein comprising an amino acid sequence of SEQ ID NO:24. In yet another embodiment, the FGF proteins in the formulation comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, (5) a protein comprising an amino acid sequence of SEQ ID NO:32, (6) a carbamylated protein comprising an amino acid sequence of SEQ ID NO:24, and (7) a carbamylated protein comprising an amino acid sequence of SEQ ID NO:2.

The present invention provides improved formulations comprising arginine in a salt form, sodium phosphate monobasic (NaH₂PO₄.H₂O), a surfactant, and one or more CG53135 proteins. In one embodiment, the present invention provides improved formulations comprising 0.1-1 M arginine in a salt form, 0.01-0.1 M sodium phosphate monobasic (NaH₂PO₄.H₂O), 0.01%-0.1% weight/volume (“w/v”) polysorbate 80 or polysorbate 20, and 0.005-50 mg/ml of one or more CG53135 proteins. The arginine in a salt form, sulfobutyl ether Beta-cyclodextrin sodium, or sucrose thereof can be, but is not limited to, arginine, arginine sulfate, arginine phosphate, and arginine hydrochloride. In a preferred embodiment, arginine sulfate is used. In some embodiments, the final concentration of the arginine in a salt form, sulfobutyl ether Beta-cyclodextrin sodium, or sucrose thereof is 0.01-0.7 M. In one embodiment, the final concentration of the arginine in a salt form, sulfobutyl ether Beta-cyclodextrin sodium, or sucrose thereof is 0.5 M. In some embodiments, the concentration of sodium phosphate monobasic in the formulations is between 0.02-0.09 M, 0.03-0.08 M, or 0.04-0.06M. In a specific embodiment, the sodium phosphate monobasic is 0.05M. In one embodiment, the improved formulations comprise a surfactant, which may be added, e.g., during the diafiltration and/or ultrafiltration step, to minimize the formation of aggregates. The surfactant can be, but is not limited to, polysorbate 80 and polysorbate 20. In a specific embodiment, the concentration of polysorbate 80 or polysorbate 20 is 0.01% (weight/volume).

The improved formulations of the present invention comprise one or more CG53135 proteins. In one embodiment, the improved formulations of the invention comprise one or more proteins comprising an amino acid sequence selected from the group consisting of SEQ ID NOs:2, 4, 7, 10, 12, 14, 16, 18, 20, 22, 24, 26, 28, 30, 32, 34, 36, 38 and 40. In another embodiment, the improved formulations of the invention comprise one or more proteins comprising an amino acid sequence selected from the group consisting of SEQ ID NOs:2, 4, 7, 10, 22, 24, 26, 28, 30, 32, 34, 36, 38 and 40. In a specific embodiment, the improved formulations of the invention comprise a protein comprising an amino acid sequence of SEQ ID NO:2. In another specific embodiment, the improved formulations of the invention comprise a protein comprising an amino acid sequence of SEQ ID NO:24. In yet another specific embodiment, the improved formulations of the invention comprise (1) a protein comprising an amino acid sequence of SEQ ID NO:2, and (2) a protein comprising an amino acid sequence of SEQ ID NO:24. In a specific embodiment, the improved formulations of the invention comprise (1) a protein comprising an amino acid sequence of SEQ ID NO:2, (2) a protein comprising an amino acid sequence of SEQ ID NO:24, (3) a protein comprising an amino acid sequence of SEQ ID NO:26, (4) a protein comprising an amino acid sequence of SEQ ID NO:28, (5) a protein comprising an amino acid sequence of SEQ ID NO:30, and (6) a protein comprising an amino acid sequence of SEQ ID NO:32. In one embodiment, the improved formulations of the invention comprise one or more proteins produced by any of the processes described in Section 5.2, supra. In some embodiments, the concentration of one or more CG53135 proteins in the improved formulations of the instant invention is at least 2 mg/ml, at least 10 mg/ml, at least 15 mg/ml, at least 20 mg/ml, at least 25 mg/ml, at least 30 mg/ml, at least 35 mg/ml, at least 40 mg/ml, at least 45 mg/ml, or at least 50 mg/ml. In some embodiments, the concentration of one or more CG53135 proteins in the improved formulations of the instant invention is no more than 50 mg/ml, no more than 30 mg/ml, no more than 10 mg/ml, no more than 5 mg/ml, no more than 1 mg/ml, or no more than 0.5 mg/ml. In some embodiments, the concentration of one or more CG53135 proteins in the improved formulations of the instant invention is 0.0005-60 mg/ml, 0.005-50 mg/ml, 0.05-50 mg/ml, 0.5-50 mg/ml, 1-60 mg/ml, 1-50 mg/ml, 5-40 mg/ml, 5-30 mg/ml, or 5-20 mg/ml. In a specific embodiment, the concentration of one or more CG53135 proteins in the improved formulations of the instant invention is 10 mg/ml.

The improved formulations of the invention can be lyophilized or spray dried, which results more stable products with longer shelf life and the ease of handling and shipment. The process of lyophilization is very well known in the art and is not described in detail herein. Briefly, lyophilization is the process by which the moisture content of the product is reduced by freezing and subsequent sublimation under vacuum. The lyophilization process primarily consists of three stages. The first stage involves freezing the product and creating a frozen matrix suitable for drying. This step impacts the drying characteristics in the next two stages. The second stage is primary drying. Primary drying involves the removal of the ice by sublimation by reducing the pressure (to typically around 50-500 μm Hg) of the product's environment while maintaining the product temperature at a low, desirable level. The third stage in the process is called secondary drying where the bound water is removed until the residual moisture content reaches below the target level. Any lyophilization process known in the art can be used to lyophilize the formulations of the invention.

The objective of a lyophilization process is to achieve a freeze-dried protein cake with acceptable appearance, biological potency, ease of reconstitution, and long-term storage stability. A prudently designed lyophilization cycle is one that is robust, consumes less time and energy, and maintains product quality. Both formulation-related and cycle-related factors contribute to achieving this goal.

The addition of a lyophilization excipient in the processes described herein may be necessary. One or more excipients may be added. The lyophilization excipients contemplated for use in the present processes include, but are not limited to, sucrose, lactose, mannitol, dextran, sucrose, heparin, glycine, glucose, glutamic acid, gelatin, sorbitol, histidine, dextrose, trehalose, methocel, hydroxy ethyl cellulose, hydroxy ethyl starch, poly(ethylene glycol), poly(vinyl pyrolidone), sulfobutyl ether Beta-cyclodextrin sodium and polyvinyl alcohol, or various combinations thereof, as well as other buffers, protein stabilizers, cryoprotectants, and cryopreservatives commonly used by those skilled in the art.

Since the active ingredient of the improved formulations of the invention is a FGF protein, preferably one or more CG53135 protein, the improved formulations of the invention can be used accordingly in any situation that a FGF protein, preferably a CG53135 protein, is known to be effective. For example, the improved formulations of the invention can be used in prevention and/or treatment of disorders such as alimentary mucositis, inflammatory bowel disease, osteoarthritis, disorders of the central nerve system or cardiovascular system, and disorders associated with radiation exposure or symptoms thereof.

### 6. EXAMPLE

Certain embodiments of the invention are illustrated by the following non-limiting examples.

### 6.1 Example 1

### Expression of CG53135

Several different expression constructs were generated to express a CG53135 protein (Table 1).

TABLE 1
Constructs Generated to Express CG53135

| Construct | Construct Description | Construct Diagram |
|---|---|---|
| 1a | NIH 3T3 cells were transfected with pFGF-20, which incorporates an epitope tag (V5) and a polyhistidine tag into the carboxy-terminus of the CG53135-01 protein in the pcDNA3.1 vector (Invitrogen) | 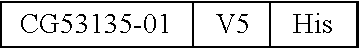 |
| 1b | Human 293-EBNA embryonic kidney cells or NIH 3T3 cells were transfected with CG53135- 01 using pCEP4 vector (Invitrogen) containing an lgK signal sequence, multiple cloning sites, a V5 epitope tag, and a polyhistidine tag | 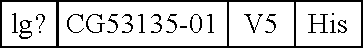 |
| 2 | E.coli BL21 cells were transformed with CG53135- 01 using pETMY vector (CuraGen Corporation) containing a polyhistidine tag and a T7 epitope tag (this construct is also referred to as E.coli/ pRSET) | 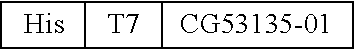 |
| 3 | E.coli BLR (DE3) cells (NovaGen) were transformed with CG53135- 05 (full-length, codon- optimized) using pET24a vector (NovaGen) | 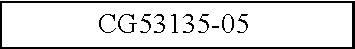 |
| 4 | E.coli BLR (DE3) cells (NovaGen) were transformed with CG53135 (deletion of amino acids 2-54, codon-optimized) using pET24a vector (NovaGen) | 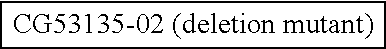 |

In one construct, CG53135-01 (the full-length CG53135 gene) was cloned as a Bgl II-Xho I fragment into the Bam HI-Xho I sites in mammalian expression vector, pcDNA3.1V5His (Invitrogen Corporation, Carlsbad, Calif.). The resultant construct, pFGF-20 (construct 1a) has a 9 amino acid V5 tag and a 6 amino acid histidine tag (His) fused in-frame to the carboxy-terminus of CG53135-01.

These tags aid in the purification and detection of CG53135-01 protein. After transfection of pFGF-20 into murine NIH 3T3 cells, CG53135-01 protein was detected in the conditioned medium using an anti-V5 antibody (Invitrogen, Carlsbad, Calif.).

The full-length CG53135-01 gene was also cloned as a Bgl II-Xho I fragment into the Bam HI-Xho I sites of mammalian expression vector pCEP4/Sec (CuraGen Corporation). The resultant construct, plg?-FGF-20 (construct 1b) has a heterologous immunoglobulin kappa (IgK) signal sequence that could aid in secretion of CG53135-01. After transfection of plgK-FGF-20 into human 293 EBNA cells (Invitrogen, Carlsbad, Calif.; catalog # R620-07), CG53135-01 was detected in the conditioned medium using an anti-V5 antibody.

In order to increase the yield of CG53135 protein, a Bgl II-Xho I fragment encoding the full-length CG53135-01 gene was cloned into the Bam HI-Xho I sites of E. coli expression vector, pETMY (CuraGen Corporation). The resultant construct, pETMY-FGF-20 (construct 2) has a 6 amino acid histidine tag and a T7 tag fused in-frame to the amino terminus of CG53135. After transformation of pETMY-FGF-20 into BL21 E. coli (Novagen, Madison, Wis.), followed by T7 RNA polymerase induction, CG53135-01 protein was detected in the soluble fraction of the cells.

In order to express FGF-20 without tags, CG53135-05 (a codon-optimized, full-length FGF-20 gene) and CG53135-02 (a codon-optimized deletion construct of FGF-20, with the N-terminal amino acids 2-54 removed) were synthesized. For the full-length construct (CG53135-05), an Nde I restriction site (CATATG) containing the initiator codon was placed at the 5′ end of the coding sequence. At the 3′ end, the coding sequence was followed by 2 consecutive stop codons (TAA) and a Xho restriction site (CTCGAG). The synthesized gene was cloned into pCRScript (Stratagene, La Jolla, Calif.) to generate pCRScript-CG53135-05. An Nde I-Xho I fragment containing the codon-optimized CG53135-05 gene was isolated from the pCRscript-CG53135-05 and subcloned into Nde I-Xho I-digested pET24a to generate pET24a-CG53135-05 (construct 3). The full-length, codon-optimized version of FGF-20 is referred to as CG53135-05.

To generate a codon-optimized deletion construct for CG53135, oligonucleotide primers were designed to amplify the truncated FGF-20 gene from pCRScript-CG53135-05. The forward primer contained an Nde I site (CATATG) followed by coding sequence starting at amino acid 55. The reverse primer contained a HindIII restriction site. A single PCR product of approximately 480 base pairs was obtained and cloned into pCR2.1 vector (Invitrogen) to generate pCR2.1-CG53135del. An Nde 1-Hind III fragment was isolated from pCR2.1-53135del and subcloned into Nde 1-Hind III-digested pET24a to generate pET24a-CG53135-02 (construct 4).

The plasmids, pET24a-CG53135-05 (construct 3) and pET24a-CG53135-02 (construct 4) have no tags. Each vector was transformed into E. coli BLR (DE3), induced with isopropyl thiogalactopyranoside. Both the full-length and the N-terminally truncated CG53135 protein was detected in the soluble fraction of cells.

### 6.2 Example 2

### Manufacture of CG53135 Drug Product for Clinical Uses (Process 2)

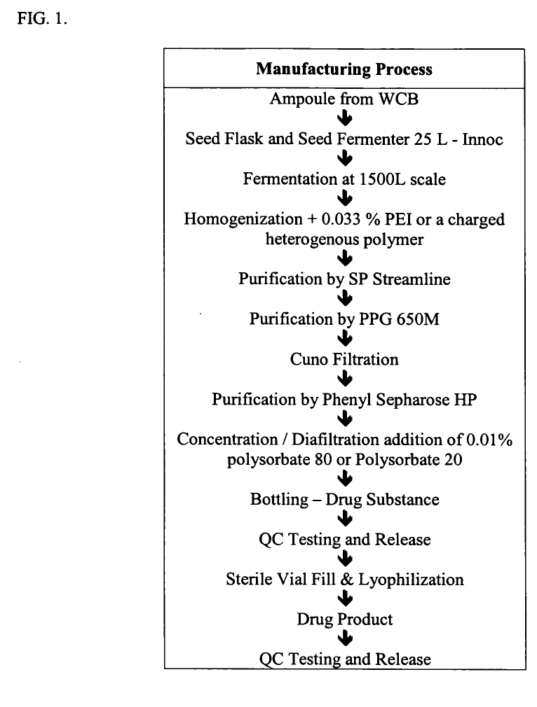

An improved manufacture process for producing a drug product comprising one or more CG53135 proteins for clinical uses has been developed. FIG. 1 shows the steps involved in the improved manufacturing process of CG53135. The codon-optimized, full-length, untagged molecule of CG53135-05 (construct 3 in Example 1) was used. The process steps for the improved manufacture process are described below.

Cell Bank: a Manufacturing Master Cell Bank (MMCB) in animal component free complex medium was used in an earlier Process. A second Manufacturing Master Cell Bank (MMCB) in animal component free chemically defined medium was derived from the first MMCB and a Manufacturing Working Cell Bank (MWCB) was made from the second MMCB. This MWCB was used in the manufacturing process as described in FIG. 1.

Innoculum Preparation: the initial cell expansion occurs in shake flasks. Seed preparation is done by thawing and pooling 2-3 vials of the MWCB in chemically defined medium and inoculating 3-4 shake flasks each containing 500 mL of chemically defined seed medium.

Seed and Final Fermentation: the shake flasks with cells in exponential growth phase (2.5-4.5 OD₆₀₀ units) are used to inoculate a single 25 L (i.e., working volume) seed fermenter containing the seed medium. The cells upon reaching exponential growth phase (3.0-5.0 OD₆₀₀ units) in the 25 L seed fermenter are transferred to a 1500 L production fermenter with 780-820 L of chemically defined batch medium. During fermentation, the temperature is controlled at 37±2° C., pH at 7.1±0.1, agitation at 150-250 rpm and sparging with 0.5-1.5 (vvm) of air or oxygen-enriched air to control dissolved oxygen at 25% or above. Antifoam agent (Fermax adjuvant 27) is used as needed to control foaming in the fermenter. When the OD (at 600 nm) of culture reaches 25-35 units, additional chemically defined medium is fed at 0.7 g/kg broth/min initially and then with feed rate adjustment as needed. The induction for expression of the CG53135-05 protein is started when OD at 600 nm reaches 135-165 units. After 4 hours post-induction the fermentation is completed. The final fermentation broth volume is approximately 1500 L. The culture is then chilled to 10-15° C.

Homogenization: the chilled culture is diluted with cell lysis buffer at the ratio of one part of fermentation broth to two parts of cell lysis buffer (50 mM sodium phosphate, 60 mM EDTA, 7.5 mM DTT, 4.5 M urea, pH 7.2. Polyethyleneimine (PEI), a flocculating agent is added to the diluted fermentation broth to a final PEI concentration at 0.033% (WN). The cells are lysed at 10-15° C. with 3 passages through a high-pressure homogenizer at 750-850 bar.

Capture and Recovery: the chilled cell lysate is directly loaded in the upflow direction onto a pre-equilibrated Streamline SP expanded bed cation exchange column. During the loading, the bed expansion factor is maintained between 2.5-3.0 times the packed bed column volume. After loading, the column is flushed with additional Streamline SP equilibration buffer (100 mM sodium phosphate, 40 mM EDTA, 10 mM sodium sulfate, 3 M urea, pH 7.0) in the upflow direction. The column is then washed further with SP Streamline wash buffer (100 mM sodium phosphate, 5 mM EDTA, 25 mM sodium sulfate, 2.22 M dextrose, pH 7.0) in the downflow direction. The protein is eluted from the column with Streamline SP elution buffer (100 mM sodium phosphate, 5 mM EDTA, 200 mM sodium sulfate, 1 M L-arginine, pH 7.0) in the downflow direction.

PPG 650M Chromatography: the SP Streamline eluate is loaded on to a pre-equilibrated PPG 650 M, hydrophobic interaction chromatography column. The column is equilibrated and washed with 100 mM sodium phosphate, 200 mM sodium sulfate, 5 mM EDTA, 1 M Arginine pH 7.0. The column is further washed with 100 mM sodium phosphate, 5 mM EDTA, 0.9 M Arginine, pH 7.0. The product is eluted with 100 mM sodium phosphate, 5 mM EDTA, 0.2 M Arginine, pH 7.0.

CUNO Filtration: the PPG eluate is passed through an endotoxin binding CUNO 30ZA depth filter. The filter is flushed first with water for injection (WFI) and then with 100 mM sodium phosphate, 5 mM EDTA, 0.2 M Arginine, pH 7.0 (PPG eluate buffer). After flushing, the PPG eluate is passed through the filter. Air pressure is used to push the final liquid through the filter and its housing.

Phenyl Sepharose Chromatography: the CUNO filtrate is then loaded on to a pre-equilibrated Phenyl Sepharose hydrophobic interaction chromatography column. The column is equilibrated and washed with 100 mM sodium phosphate, 50 mM ammonium sulfate, 800 mM sodium chloride, 0.5 M Arginine, pH 7.0. The product is eluted with 50 mM sodium phosphate, 0.5 M Arginine, pH 7.0.

Concentration and Diafiltration: a 1% Polysorbate 80 is added to the Phenyl Sepharose eluate so that the final concentration in the drug substance is 0.01% (w/v). The eluate is then concentrated in an ultrafiltration system to about 2-3 g/L. The retentate is then diafiltered with 7 diafiltration volumes of 50 mM sodium phosphate, 0.5 M Arginine, pH 7.0 (Phenyl Sepharose elution buffer). After diafiltration the retentate is concentrated between 12-15 g/L. The retentate is filtered through a 0.22 μm filter and subsequently diluted to 10 g/L.

Bulk Bottling: the retentate from the concentration and diafiltration step is filtered through a 0.22 μm pore size filter into 2 L single use teflon bottles. The bottles are frozen at −70° C.

Drug Product/Vial: the Frozen Drug Substance is used for the manufacture of the Drug Product. The bottles of frozen Drug Substance are thawed at ambient temperature. After the Drug Substance is completely thawed, it is pooled in a sterile container, filtered, filled into vials, partially stoppered, and lyophilized. After completion of the freeze-drying process, the vials are stoppered and capped. The lyophilized Drug Product is stored at 2-8° C.

The above described method (Process 2) is an improved method over a production method (Process 1) described in U.S. patent application Ser. No. 10/435,087, filed May 9, 2003, the content of which is incorporated herein by reference. Briefly, Process 1 can be described as follows:

Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 30-40 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100˜110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 uM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at ˜80±15° C.

### 6.3 Example 3

### Charaterization of Reference Standard of the Drug Product

A protein reference standard was prepared using a 140 L scale manufacturing process that was representative of the bulk drug substance manufacturing process as described in Section 6.2 (Example 2). The reference standard was stored as 1 mL aliquots in 2 mL cryovials at −80° C.±15° C. The proposed specifications for the reference standard are listed in Table 2.

TABLE 2
Proposed Specifications of CG53135-05 Reference Standard

| Property | Assay | Description of Expected Results |
|---|---|---|
| Purity | SDS-PAGE (reduced, colloidal | >98% pure by densitometry |
| | Coomassie stain) | |
| | SDS-PAGE (reduced, silver stain) | At 10 μg load, less than 100 ng of |
| | | impurities are detectable |
| | SEC-HPLC | FIO* |
| | RP-HPLC | ≧90% main peak |
| | Host cell protein Western Blot | As found |
| Identity | Western Blot | Major band ˜23 kDa |
| | N-Terminal amino acid sequencing | Consistent with predicted primary N- |
| | | terminal amino acid sequence |
| | LC and MS | HPLC profile shows 1 major peak |
| | | which is confirmed by MS to be |
| | | CG53135-related |
| | Total amino acid analysis | Consistent with predicted primary |
| | | amino acid composition |
| | Peptide mapping | Fragment pattern consistent with |
| | | predicted primary amino acid sequence |
| Strength | Total protein by A₂₈₀ | 10 ± 1 mg/mL |
| Potency | Bioassay (Relative Potency) | 60-140% relative to reference |
| | | standard |
| Secondary | Far-UV CD spectroscopy | As found |
| Structure | | |
| Tertiary | UV derivative spectroscopy | As found |
| Structure | Near-UV CD spectroscopy | As found |
| Quartenary | Differential Scanning Calorimetry | As found |
| Structure | Light Scattering (SEC-HPLC) | As found |
| Safety | Residual DNA | ≦100 pg/mg |
| | Endotoxin (USP <85> gel clot) | ≦2 EU/mg |
| | Bioburden | <1 cfu/mL |
| Other | pH | 7.0 ± 0.5 |
| | Osmolarity | As found |
| | Sulfyhydryl content | As found |
| | Visual inspection | Clear and colorless solution |

*FIO: for information only.

### 6.4 Example 4

### Purity Analysis of the Drug Product

The drug product produced by the manufacturing process as described in Section 6.2 (Example 2) is analyzed for its purity by the experiments described in this section.

6.4.1 Purity by SDS-PAGE Analyses

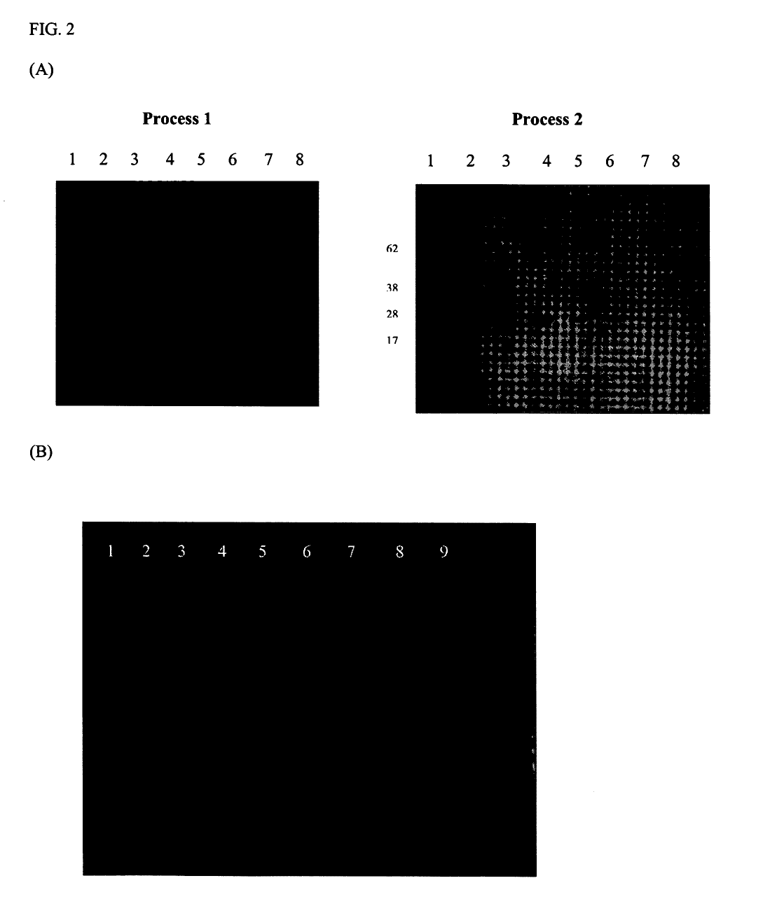

Purified protein using the improved manufacturing process as described in Section 6.2 (“Process 2”) and the purified protein using the previous method as described in U.S. application Ser. No. 10/435,087 (“Process 1”) were analyzed by loading increasing amounts of protein on a 4-12% gradient Bis-Tris NuPAGE gel and stained with the gel code blue stain to detect trace impurities (FIG. 2A). Purified protein (from both Process 1 and Process 2, respectively) migrated as a single major band under reducing conditions (˜23 kDa). No impurities above the LOD (<28 ng) were detected.

Purified protein of Process 1 and Process 2 was also analyzed by loading increasing amounts of protein on a 4-12% gradient Bis-Tris NuPAGE gel and using silver stain to detect trace impurities (FIG. 2B). Purified protein (from both Process 1 and Process 2, respectively) migrated as a single major band (˜23 kDa) at all loads under reducing conditions.

6.4.2 Purity by RP-HPLC

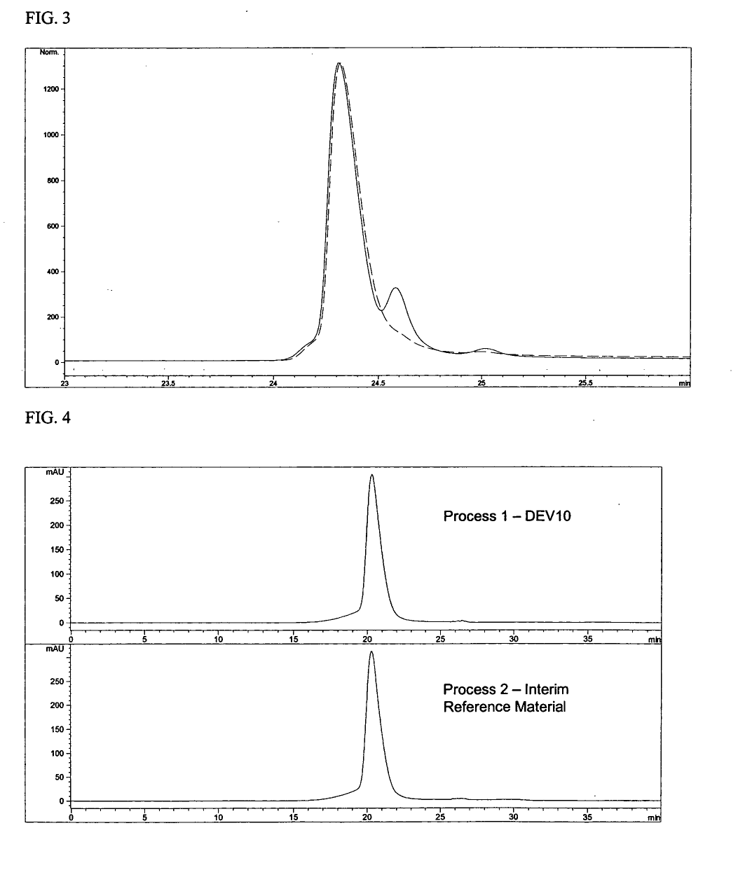

Purified drug product was analyzed by reversed-phase high-performance liquid chromatography (RP-HPLC). Purified protein from Process 1 and Process 2 was loaded onto a Protein C4 column (Vydac, 5 μm, 150 mm×4.6 mm) using a standard HPLC system in a mobile phase containing water, acetonitrile and trifluoroacetic acid. Purified protein from Process 1 elutes as a major peak at 24.0 min and additional peaks at 24.3 and 24.7 minutes. These represent isoforms of CG53135-05. CG53135-05 obtained using Process 2 elutes as a major peak with a retention time of 24.0 minutes (FIG. 3). Characterization of these peaks is discussed further below in Section 6.7 (Example 7).

6.4.3 Purity by Size Exclusion-HPLC

Purified protein (from both Process 1 and Process 2, respectively) was analyzed by size exclusion chromatography (SEC-HPLC) with UV detection at 280 nm. Analysis was performed by injecting the protein onto a size exclusion HPLC column (Bio-Sil SEC-250, 0.78 cm×30 cm, Bio-Rad) using a standard HPLC system with a mobile phase containing 100 mM sodium phosphate, 1 M arginine-HCl, pH 7.0. Purified protein eluted isocratically as a single mono-disperse peak with a retention time of 20.5 minutes (FIG. 4) for Process 1 and 2. This retention time corresponds to an apparent molecular weight of approximately 45 kilodaltons (when compared against a set of calibration standards run under identical conditions), which suggests that FGF-20 exists as a non-covalently linked dimer.

6.4.4 Host Cell Protein Determination via Western Blot

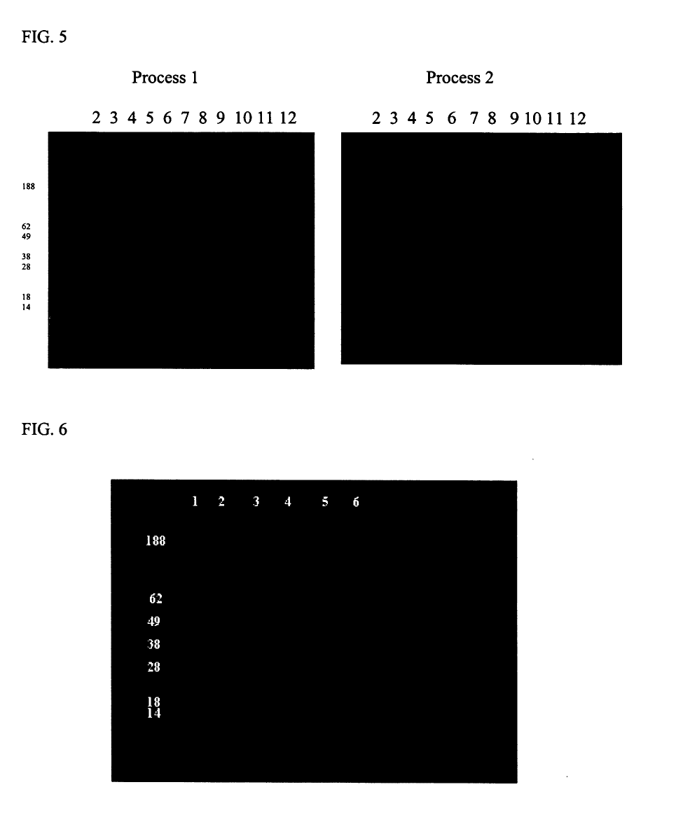

The levels of host cell protein impurities in purified drug product were assessed qualitatively by Western blot analysis. The purified CG53135 protein was resolved by SDS-PAGE and electrophoretically transferred to a nitrocellulose membrane. The membrane was incubated with a primary antibody (rabbit anti-E. coli, Dako Systems) followed by a secondary antibody (goat anti-rabbit Alkaline Phosphatase conjugated, Bio-Rad) and developed using standard techniques. No host cell protein impurities were visible for Process 1 and only one band (−70 kDa) is apparent from Process 2 (FIG. 5).

6.4.5 Identity of CG53135 via Western Blot

Purified protein (from both Process 1 and Process 2, respectively) was identified by Western blot using rabbit polyclonal anti-CG53135 sera (FIG. 6). Purified CG53135-05 was resolved by loading 10 μg of protein on a 4-12% gradient Bis-Tris NuPAGE gel and electrophoretically transferred to a nitrocellulose membrane. The membrane was incubated with a primary antibody (polyclonal anti-CG53135 sera) followed by a secondary antibody (goat anti-rabbit Alkaline Phosphatase conjugated, Bio-Rad) and then developed using standard techniques. Purified protein (from both Process 1 and Process 2, respectively), which migrates as a single band of the expected molecular weight (molecular weight of FGF-20) under reducing and nonreducing conditions, is immunoreactive with CG53135-specific antiserum.

### 6.5 Example 5

### Potency of CG53135 Product

The potency was measured by cell growth of NIH 3T3 cells in response to the purified protein from Process 1 and Process 2. Cell growth was measured indirectly using fluorescence by the conversion of resazurin (CellTiter Blue Reagent) into resorufin. Using DEV-10 (Process 1) as the reference standard, the Process 2 interim reference standard was found to have comparable potency at 101%. Several lots manufactured by Process 2 were analyzed. These results are shown in Table 3.

TABLE 3
Potency of Lots from Process 2 using DEV-10
(Process 1) as Reference Standard

| Lot Number | Potency Result (%) |
|---|---|
| PHP 020904-1 | 113 |
| PHP 020904-2 | 123 |
| CUNO-0104-1 | 102 |
| CUNO-0204-1 | 95 |
| PT 0504A | 114 |
| PT 0504B | 98 |
| DS 1002-01 | 100 |

The average potency for all of the lots tested is 106.4±10.3. This indicates that the potency of lots from Process 2 are equivalent to lot DEV-10 made with Process 1. Residual DNA, endotoxin and bioburden in the drug substance can also be tested using qualified assays.

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was not included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 4). The PI₂₀₀ was defined as the concentration of protein that resulted in incorporation of BrdU at 2 times the background. CG53135-05 and CG53135-05 related species recovered from all 3 measurable peaks demonstrated similar biological activity with a P1200 of 0.7-11 ng/mL (Table 4).

TABLE 4
Biological Activity of CG53135-05 E. coli purified product
(DEV10): Induction of DNA Synthesis

| PI₂₀₀ (ng/mL) | CG53135-05 (DEV 10) | Peak 1 | Peak 2 | Peak 3 |
|---|---|---|---|---|
| | 1.0 | 0.7 | 11 | 8.6 |

### 6.6 Example 6

### Characterization Studies for Biochemical Comparability

Characterization studies for comparison of the primary, secondary, tertiary and quaternary structure as stated below in Table 5 may also be done. The side-by-side results of reference standard (designated DEV10) from the Process 1 with reference standard obtained using Process 2 (interim reference material) can be used to further demonstrate the biochemical properties of the purified CG53135 protein.

TABLE 5
Biochemical Characterization of CG53135-05 Reference Materials

| Attributes | Process 1 | Process 2 |
|---|---|---|
| Purity | SDS-PAGE (silver) | SDS-PAGE (silver) |
| | HCP Western | (HCP Western - release assay) |
| Identity | Western | RP HPLC - peak |
| | | identification |
| (Primary | N-Terminal sequencing | N-Terminal sequencing |
| Structure) | Total Amino acid analysis | Total Amino acid analysis |
| | Peptide mapping | Peptide mapping/LC-MS |
| | MALDI-TOF MS | |
| Secondary | | Far UV CD spectroscopy |
| Structure | | |
| Tertiary | | UV-derivative spectroscopy |
| Structure | | Near UV CD spectroscopy |
| Quartenary | | Light Scattering (SEC-HPLC) |
| Structure | | Diff. scanning calorimetry |
| Other | pH | pH |
| | Osmolality | Osmolality |
| | Sulfhydryl content | Sulfhydryl content |

### 6.7 Example 7

### Characterization of Primary Structure of the Purified Protein

6.7.1 RP-HPLC Assay: Peak Identification

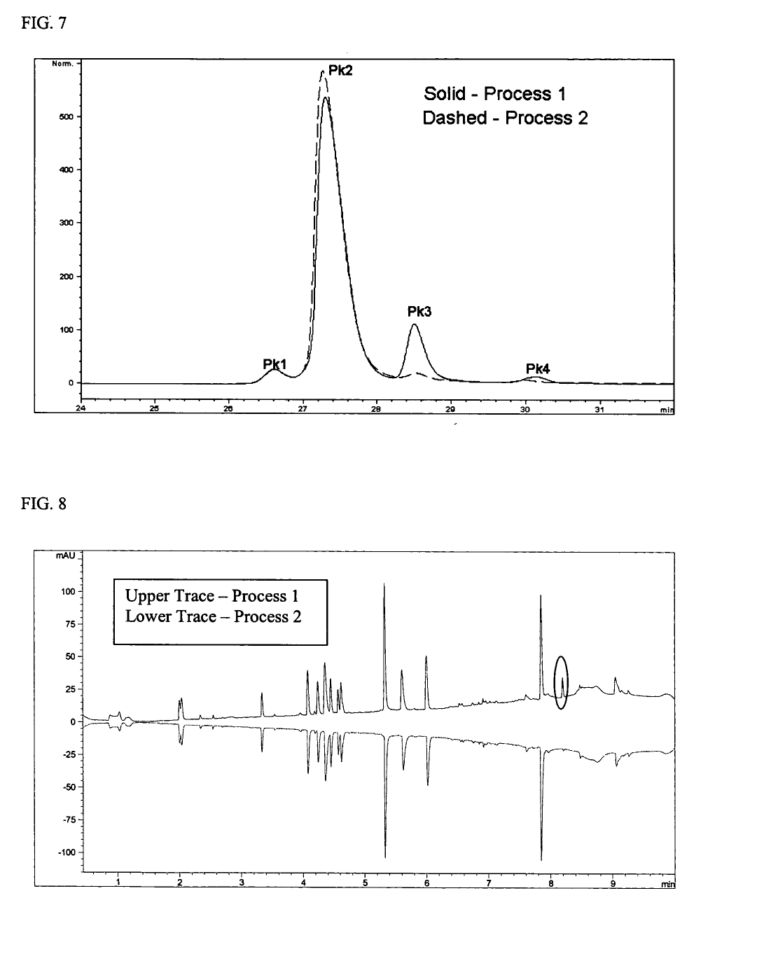

Purified drug substance (by both Process 1 and Process 2, respectively) was further analyzed by reversed-phase high-performance liquid chromatography (RP-HPLC) with both UV and electrospray mass spectrometric detection. Purified protein from either Process 1 or Process 2 was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 7). These peaks were characterized by electrospray mass spectrometry. As can be observed from the chromatograms, the four equipotent peaks are present in the purified final product from Process 1 and 2. However, the proportion of these peaks (1, 3 and 4) is much lower in the final product purified by Process 2 with the predominant form being Peak 2.

The identities of each peak from the RP-HPLC separation are indicated in Table 6.

TABLE 6
Identity of peaks from the RP-HPLC separation of CG53135-05
based upon accurate molecular weight determination.

| | Reten- | | | | |
|---|---|---|---|---|---|
| | tion | Molecular | | | Predicted |
| Peak | Time | Weight | Assignment | | Molecular |
| # | (min) | Observed | (residue #) | ID Number | Weight |
| 1 | 26.6 | 21329.2 | 24-211 | CG53135-17 | 21329.2 |
| 1 | 26.6 | 22185.1 | 15-211 | CG53135-16 | 22185.1 |
| 1 | 26.6 | 22412.4 | 12-211 | CG53135-15 | 22412.4 |
| 2 | 27.3 | 23296.5 | 3-211 | CG53135-13 | 23296.4 |
| 3 | 28.5 | 23498.9 | 1-211 | CG53135-05 | 23498.7 |
| 4 | 30.0 | 23339.3 | 3-211 | CG53135-13 | 23339.4 |
| | | | (carbamylated) | (carbamylated) | |
| 4 | 30.0 | 23539.7 | 1-211 | CG53135-05 | 23539.7 |
| | | | (carbamylated) | (carbamylated) | |

6.7.2 Edman Sequencing and Total Amino Acid Analysis

The experimental N-terminal amino acid sequence of the Process 1 reference standard, DEV 10, and the Process 2 interim reference standard were determined qualitatively. The reference standards were resolved by SDS-PAGE and electrophoretically transferred to a polyvinylidenefluoride membrane; the Coomassie-stained ˜23 kDa major band corresponding to each reference standard was excised from the membrane and analyzed by an automated Edman sequencer (Procise, Applied Biosystems, Foster City, Calif.). A comparison of the two major sequences is shown in Table 7 below. The predominant sequence for each reference standard were identical and corresponded to residues 3-20 in the theoretical N-terminal sequence of CG53135-05.

TABLE 7
Edman sequencing data for the first 20 amino acids of
CG53135-05 for Process 1 and 2.

| | |
|---|---|
| Theoretical Residue | Amino Acid Residue |

| Position | Process 1 | Process 2 |
|---|---|---|
| 3 | Pro | Pro |
| 4 | Leu | Leu |
| 5 | Ala | Ala |
| 6 | Glu | Glu |
| 7 | Val | Val |
| 8 | Gly | Gly |
| 9 | Gly | Gly |
| 10 | Phe | Phe |
| 11 | Leu | Leu |
| 12 | Gly | Gly |
| 13 | Gly | Gly |
| 14 | Leu | Leu |
| 15 | Glu | Glu |
| 16 | Gly | Gly |
| 17 | Leu | Leu |
| 18 | Gly | Gly |
| 19 | Gln | Gln |
| 20 | Gln | Gln |

The experimental amino acid composition of the DEV10 reference standard and the PX3536G001-H reference standard were determined in parallel. Quadruplicate samples of each reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μL sample buffer containing 2 nmol homoserine as an internal standard. The amino acids in each sample were separated on a Beckman Model 7300 amino acid analyzer. The amino acid composition of both reference standards showed no significant differences as shown in Table 8 below. Note that Cys and trp are destroyed during acid hydrolysis of the protein. Asn and gln are converted to asp and glu, respectively, during acid hydrolysis and thus their respective totals are reported as asx and glx. Met and his were both unresolved in this procedure.

TABLE 8
Quantitive amino acid analysis for final purified protein
from Process 1 and Process 2

| | |
|---|---|
| Amino Acid | Mole Percent |

| Residue | DEV10 | PX3536G001-H |
|---|---|---|
| asx | 7.1 | 7.0 |
| thr | 4.0 | 4.0 |
| ser | 6.3 | 6.1 |
| glx | 12.2 | 12.2 |
| pro | 6.0 | 6.0 |
| gly | 14.4 | 14.3 |
| ala | 5.8 | 5.6 |
| val | 5.3 | 5.3 |
| ile | 3.5 | 3.5 |
| leu | 13.6 | 13.6 |
| tyr | 4.6 | 4.6 |
| phe | 5.2 | 5.2 |
| lys | 3.7 | 3.7 |
| arg | 8.5 | 9.1 |

6.7.3 Tryptic Mapping by RP-HPLC

Purified drug substance from Process 1 and 2 was reduced and alklated with iodoacetic acid and then digested with sequencing grade trypsin. The tryptic peptides were separated by reversed-phase high-performance liquid chromatography (RP-HPLC) using both UV and electrospray mass spectrometric detection. The tryptic digest from either Process 1 or Process 2 was loaded onto an ODS-1 nonporous silica column (Micra, 1.5 μm; 53×4.6 mm) using a standard HPLC system in a mobile phase containing water, acetonitrile and trifluoroacetic acid. The eluting peptides were detected by UV at 214 nm (FIG. 8) and by positive-ion electrospray mass spectrometry. The major difference between the two chromatograms for Process 1 and Process 2 is the reduction in peak area of a peak obvious in the Process I trace (peak at 8.2 min; FIG. 8). This peak corresponds to the T1 peptide, residues 1-40. This observation is expected since the source of this peptide if from the intact CG53135-05, which is in greater abundance in the Process I material (peak 3, FIG. 7).

### 6.8 Example 8

### Characterization of Secondary Structure: Circular Dichoroism Spectroscopy of the Purified Protein

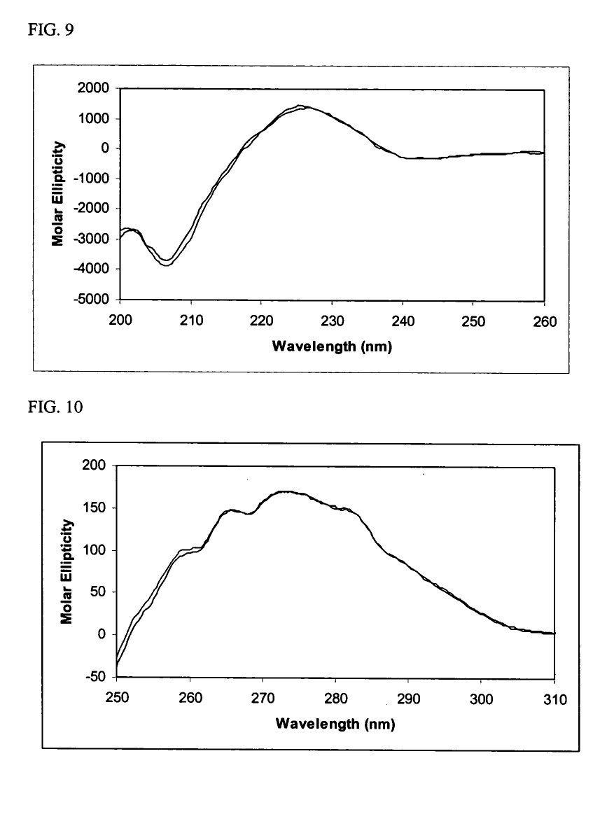

The far UV circular dichroism spectrum of the purified protein (from both Process 1 and Process 2, respectively) is characterized by a broad maximum at 226-227 nm and a sharp minimum at approximately 206 nm. Both features are common in other fibroblast growth factors and suggest a secondary structure dominated by β-sheet and β-turns. The far UV circular dichroism spectra of the DEV10 reference standard and the PX3536G001-H reference standard both display these features and are nearly identical (FIG. 9). The small differences in the spectra are attributable to experimental error.

### 6.9 Example 9

### Characterization of Tertiary Structure

6.9.1 Near UV Circular Dichroism Spectroscopy of the Purified Protein

The near UV Circular Dichroism (CD) spectrum of a protein reflects the number and orientation of the protein's aromatic amino acids. For proteins having identical numbers of aromatic amino acids any differences in their near UV CD spectra represent differences in the position and orientation of the aromatic amino acids. The position and orientation of the aromatic amino acids are a measure of a protein's tertiary structure. Hence, differences in the near UV CD spectra for proteins represent differences in tertiary structure.

The near UV CD spectra of the DEV10 reference standard and the PX3536G001-H reference standard are shown in FIG. 10. There are no significant differences between these two spectra and suggest that both reference standards have no significant differences in their tertiary structure.

6.9.2 Second Derivative UV Absorbance Spectroscopy of the Purified Protein

The UV absorbance of aromatic amino acids is influenced by the amino acid's microenvironment. Aromatic amino acids embedded within a protein are in a less polar microenvironment than surface exposed residues. This difference in polarity has a profound effect on the UV absorbance of an aromatic amino acid. Different microenvironments can shift the spectra of aromatic amino acids 4-6 nm in extreme cases. Monitoring these changes for individual proteins is done by calculating the second derivative of the protein's UV absorbance spectrum. The second derivative UV absorbance spectrum of a protein contains a number of minima that correspond to the individual aromatic amino acids. The wavelengths of these minima reflect the microenvironment of the amino acid. Therefore, changes in these minima are indicative of conformational (tertiary) changes in the protein.

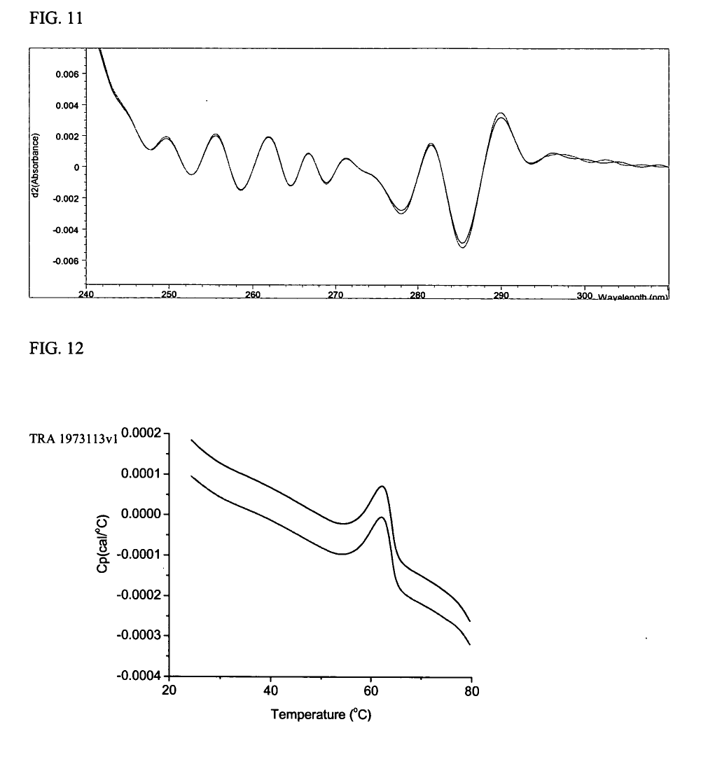

The second derivative UV absorbance spectrum of the purified protein (from both Process 1 and Process 2, respectively) is characterized by seven minima between 250 and 300 nm. As shown in Table 9 below, and qualitatively in FIG. 11, the wavelengths of all seven minima for both the DEV10 reference standard and the PX3536G001-H reference standard are not significantly different. These data demonstrate that the microenvironment around the individual aromatic amino acids in both standards are highly similar and suggests significant tertiary differences do not exist between these two reference standards.

TABLE 9
Second derivative UV absorbance spectral data for Process 1 and 2

| | | Peak 1 | Peak 2 | Peak 3 | Peak 4 | Peak 5 | Peak 6 | Peak 7 |
|---|---|---|---|---|---|---|---|---|
| Sample | Result | (Phe) | (Phe) | (Phe) | (Phe/Tyr) | (Tyr) | (Tyr/Trp) | (Trp) |
| DEV10 | Average | 252.70 | 258.60 | 264.60 | 268.90 | 277.95 | 285.38 | 293.50 |
| DEV10 | SD | 0.00 | 0.00 | 0.00 | 0.00 | 0.05 | 0.04 | 0.07 |
| PX3536G001-H | Average | 252.70 | 258.60 | 264.60 | 268.90 | 277.98 | 285.40 | 293.53 |
| PX3536G001-H | SD | 0.00 | 0.00 | 0.00 | 0.00 | 0.04 | 0.00 | 0.08 |

Results represent the average of 5 replicates for each reference standard.

### 6.10 Example 10

### Characterization of Quarternary Structure

6.10.1 Differential Scanning Calorimetry of the Purified Protein

Differential scanning calorimetric analysis is based upon the detection of changes in the heat content (enthalpy) or the specific heat of a sample with temperature. As thermal energy is supplied to the sample its enthalpy increases and its temperature rises by an amount determined, for a given energy input, by the specific heat of the sample. The specific heat of a protein changes slowly with temperature in a particular physical state, but alters discontinuously at a change of state, e.g. melting or denaturation of the protein.

As can be seen in FIG. 12 the melting curves for the purified protein are similar and the average Tm (melting temperature) is 62.25° C. for Process 1 and 62.02° C. for Process 2. These differences are within the experimental error of the instrument.

### 6.11 Example 11

### Measuring Sulfhydryl Contatent of the Purified Protein

The sulfhydryl content of the purified protein from Process 1 and Process 2 were measured (Table 10). The purified protein (from Process 1 and Process 2, respectively) was analyzed for total sulfhydryl content spectrophotometrically using 5,5′-dithio-bis (2-nitrobenzoic acid), commonly referred to as Ellman's reagent. The results indicate that the total number of measurable sulfhydryis in the final product is the same for Process 1 and Process 2 and are sufficient to account for all the theoretical sulfhydryls in the purified protein.

TABLE 10
Results from Other Characterization Assays Conducted on the
purified protein (Process 1 and Process 2, respectively)

| Assay | Process 1 | Process 2 |
|---|---|---|
| Sulfhydryl (SH) content | 108.0 ± 3.6 | 101.4 ± 4.9 |

### 6.12 Example 12

### Improved Formulations Comprising CG53135

A new formulation was developed to meet the three requirements for a commercial product: (1) the minimal storage temperature should be 2-8° C. for ease of distribution; (2) product should be stable at the storage temperature for at least 18 months for a commercial distribution system; and (3) product should be manufactured by commercial scale equipment, and processes should be transferable to various commercial contract manufacturers.

The new formulation consists 10 mg/mL of the protein product produced by the process described in Section 6.2 (“Process 2 protein”) in 0.5 M arginine as sulfate salt, 0.05 M sodium phosphate monobasic, and 0.01% (w/v) polysorbate 80. The lyophilized product is projected to be stable for at least 18 months at 2-8° C. based on accelerated stability data. In contrast to the new formulation, the previous formulation as described in U.S. application Ser. No. 10/435,087 is not possible to be lyophilized for the following reasons: firstly, the acidic component of the acetate buffer is acetic acid, which sublimes during lyophilization. This loss of acetic acid to lyophilization increases the pH to >7.5, which is far from the target pH of 5.3. Secondly, the glycerol has a collapse temperature of <−45° C., which renders this formulation not be able to be lyophilized commercially. Most of the commercial lyophilizers have a shelf temperature ranged from −45° C. to −50° C. with temperature variation of ±3° C.

Four unexpected properties of CG53135 were discovered and used to develop the new formulation: (1) high concentration of arginine, >0.4 M, increases the solubility to >30 mg/mL; (2) the use of sulfate salt of arginine increases the solubility by at least 2-6 folds; (3) the optimal concentration of sodium phosphate as a buffering salt is 50 mM, with a solubility of at least 1-2 fold increase in comparison with concentrations at 25, 75, and 100 mM; and (4) adding a surfactant during the diafiltration/ultrafiltration step minimizes the formation of aggregates. In development the lyophilized formulation, each component of the new formulation was evaluated for solubility individually. CG53135-05 was precipitated using the precipitate buffer (50 mM NaPi, 5 mM EDTA, 1 M L-Arginine HCl, 2.5 M (NH4)₂SO4). The precipitate was washed with 25 mM sodium phosphate buffer at pH 6.5 to remove the residual arginine and ammonium sulfate. The washed precipitate was then re-dissolved in the following respective buffers listed in the tables. The following are examples of data.

TABLE 11
High concentration of arginine, >0.4 M, increases the
solubility to >30 mg/mL

| | Solubility of Process 2 protein in mg/mL | | | | |
|---|---|---|---|---|---|
| Concenctration of | Batch | | | | |
| Arginine (M) | #1 | Batch #2 | Batch #3 | Batch #4 | Batch #5 |
| 0.05 | 0.7 | 0.6 | 0.5 | ND | ND |
| 0.10 | 1.4 | 0.6 | 1.2 | ND | ND |
| 0.15 | 2.2 | 1.6 | 2.2 | ND | ND |
| 0.20 | 3.0 | 4.7 | 4.3 | ND | ND |
| 0.30 | ND | ND | ND | 5.8 | ND |
| 0.35 | ND | ND | ND | 10.1 | ND |
| 0.40 | ND | ND | ND | 9.8 | ND |
| 0.45 | ND | ND | ND | 32.3 | ND |
| 0.50 | ND | ND | ND | 23.8* | 37 |

*The solubility was lower as there was not sufficient protein in the experiment to be dissolved.

TABLE 12
The optimal concentration of sodium phosphate as
a buffering salt is 50 mM

| Concentration of | Solubility of Process 2 protein in mg/mL | | | | |
|---|---|---|---|---|---|
| sodium phosphate | Batch | | | | |
| monobasic* | #A | Batch #B | Batch #C | Batch #D | Batch #E |
| 100 mM | 3.78 | 2.8 | 2.4 | 2.9 | 2.47 |
| 75 mM | 4.06 | 2.5 | 2.6 | 3.0 | 2.38 |
| 50 mM | 5.47 | 4.7 | 3.3 | 4.3 | 4.81 |
| 25 mM | 4.01 | 2.4 | 2.6 | 2.4 | 3.59 |

All formulation contains 0.2 M arginine.

An optimal concentration of the sodium phosphate as a buffering salt was observed (Table 12). The optimal concentration of sodium phosphate is 50 mM with a solubility of at least 1-2 fold increase in comparison with concentrations at 25, 75, and 100 Mm.

TABLE 13
The use of sulfate salt of arginine increases
the solubility by at least 1-3 folds

| | Solubility Increament of Process 2 | |
|---|---|---|
| | protein in using Arginine Sulfate | |
| | vs Arginine Phosphate in mg/mL | |
| Formulation | Batch #K | Batch #J |
| 50 mM sodium phosphate monobasic | 4.4 | 2.3 |
| and 0.15M Arginine at pH 7 | | |
| 50 mM sodium phosphate monobasic | 6.5 | 5.2 |
| and 0.15M Arginine at pH 7 | | |

Table 14 shows a need to add a surfactant during the diafiltration/ultrafiltration step to minimize the formation of aggregates. The experiment was conducted by performing the ultrafiltration/diafiltration at 2.5 mg/mL CG53135-05 in 0.2M arginine and 0.05 M sodium phosphate buffer at pH 7.0. After exchanging with 7 volumes of the final buffer (0.5M arginine and 0.05 M sodium phosphate buffer at pH 7.0), the diafiltrate is concentrated to ˜20 mg/mL. The diafiltrate is then diluted with the final buffer to ˜12.5 mg/mL and lyophilized. Polysorbate 80 is added either before or after the diafiltration to a final concentration of 0.01%.

TABLE 14
Adding a surfactant during the diafiltration/ultrafiltration step
minimizes the formation of aggregates.

| Polysorbate added | Process 2 | |
|---|---|---|
| during | protein Concentration | Turbidity |
| ultrafiltration/diafiltration | (mg/mL) | (NTU) |
| Yes | 12.5 | 20.9 |
| No | 13.0 | 4.6 |

All formulation contains 0.5 M arginine, 0.05 M sodium phosphate monobasic, and 0.01% polysorbate 80.

The new formulation has the following advantages: (1) a lyophilized product with a storage temperature of 2-8° C.; (2) a lyophilized product with a projected shelf-life of at least 18 months when stored at 2-8° C. achieve the solubility of >30 mg/mL; and (3) The lyophilized product has a collapse temperature of −30° C. which can be easily lyophilized by the commercial equipment. The interactions between arginine, sulfate, phosphate, and surfactant and CG53135 were unexpected.

### 7. EQUIVALENTS

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

What is claimed is:

1. A formulation comprising about 0.1-1 M arginine in a salt form, sulfobutyl ether Beta-cyclodextrin sodium, or sucrose, about 0.01-0.1 M sodium phosphate monobasic (NaH₂PO₄.H₂O), about 0.01%-0.1% weight/volume (“w/v”) polysorbate 80 or polysorbate 20, and an isolated fibroblast growth factor.

2. A formulation comprising about 0.01-1 M arginine in a salt form, sulfobutyl ether Beta-cyclodextrin sodium, or sucrose, about 0.01-0.1 M sodium phosphate monobasic (NaH₂PO₄H₂O), about 0.01%-0.1% weight/volume (“w/v”) polysorbate 80 or polysorbate 20, and an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

3. The formulation of claim 1 or 2, wherein said arginine in a salt form is selected from the group consisting of arginine, arginine sulfate, arginine phosphate, and arginine hydrochloride.

4. The formulation of claim 1 or 2, wherein said arginine in a salt form, sulfobutyl ether Beta-cyclodextrin sodium or sucrose is of 0.01-0.7 M.

5. The formulation of claim 1 or 2 comprising an arginine in a salt form at a concentration of 0.5 M.

6. The formulation of claim 1 or 2, wherein said sodium phosphate monobasic is 0.05 M.

7. The formulation of claim 1 or 2, wherein said polysorbate 80 or polysorbate 20 is 0.01% (w/v).

8. The formulation of claim 1 or 2 comprising polysorbate 80.

9. The formulation of claim 1 or 2 comprising polysorbate 20.

10. The formulation of claim 1 or 2, wherein said protein is at a concentration of 0.5-30 mg/ml.

11. The formulation of claim 1 or 2, wherein said protein is at a concentration of 10 mg/ml.

12. The formulation of claim 2, wherein said protein comprises an amino acid sequence of SEQ ID NO:24.

13. The formulation of claim 2, wherein said protein comprises an amino acid sequence of SEQ ID NO:2.

14. The formulation of claim 1 or 2 comprising two or more proteins.

15. The formulation of claim 14, wherein said proteins comprise a first protein comprising an amino acid sequence of SEQ ID NO:24, and a second protein comprising an amino acid sequence of SEQ ID NO:2.

16. The formulation of claim 1 or 2 is lyophilized or spray dried.

17. The formulation of claim 2, wherein said isolated protein is of about 0.005 mg/ml to about 50 mg/ml.

18. A formulation comprising about 10 mg/ml of an isolated protein comprising an amino acid sequence of SEQ ID NO:24, 0.5 M arginine sulfate, 0.05 M sodium phosphate monobasic, and 0.01% (w/v) polysorbate 80.

19. A formulation comprising about 10 mg/ml of an isolated protein comprising an amino acid sequence of SEQ ID NO:2, 0.5 M arginine sulfate, 0.05 M sodium phosphate monobasic, and 0.01% (w/v) polysorbate 80.

20. A formulation comprising 0.5 M arginine sulfate, 0.05 M sodium phosphate monobasic, 0.01% (w/v) polysorbate 80, and about 10 mg/ml of a mixture of isolated proteins, wherein said proteins comprise a first protein comprising an amino acid sequence of SEQ ID NO:24, and a second protein comprising an amino acid sequence of SEQ ID NO:2.

21. The formulation of claim 20 further comprising an isolated protein, wherein said protein comprises an amino acid sequence selected from the group consisting of SEQ ID NOs:26, 28, 30 and 32.

22. The formulation of claim 21, wherein one or more of the isolated protein is carbamylated.

23. The formulation of claim 20 further comprising a third protein comprising an amino acid sequence of SEQ ID NO:28, a fourth protein comprising an amino acid sequence of SEQ ID NO:30, and a fifth protein comprising an amino acid sequence of SEQ ID NO:32.

24. The formulation of any of claims 2, 18-23, wherein said isolated protein is at least 98% pure.

25. A method of increasing solubility of a fibroblast growth factor (“FGF”) in an aqueous solution comprising adding arginine in a salt form, sulfobutyl ether Beta-cyclodextrin sodium, or sucrose, or a combination thereof to said solution to a final concentration of 0.01-1 M.

26. The method of claim 25, wherein said fibroblast growth factor is an isolated protein comprising an amino acid sequence selected from the group consisting of SEQ ID NOs:2, 4, 7, 10, 22, 24, 26, 28, 30, 32, 34, 36, 38, and 40.

27. The method of claim 25 or 26, wherein said arginine in a salt form is selected from the group consisting of arginine, arginine sulfate, arginine phosphate, and arginine hydrochloride.

28. The method of claim 25 or 26, wherein said final concentration of arginine in a salt form is 0.01-0.7 M.

29. The method of claim 25 or 26, wherein said final concentration of arginine in a salt form is 0.5 M.

30. The method of claim 25 further comprising adding acetate, succinate, tartrate, or a combination thereof to said solution.

31. A method of increasing solubility of a FGF in a solution comprising adding acetate, succinate, tartrate or a combination thereof to said solution.

32. The method of claim 31, wherein said acetate, succinate, tartrate, or a combination thereof has a final concentration of 0.01-0.2 M in said solution.

33. A method of producing an isolated protein comprising the steps of:
(1) fermenting an E. coli cell containing a vector comprising SEQ ID NO:8;
(2) chilling the fermented culture to 10-15° C.;
(3) diluting the chilled culture with a lysis buffer comprising 50-100 mM sodium phosphate, 60 mM ethylene diamine tetraacetic acid, 7.5 mM DTT, and 3.5-5 M urea;
(4) lysing the cells in the diluted culture;
(5) loading the resultant cell lysate onto a pre-equilibrated cation exchange column, and flushing the column with a buffer comprising 50-100 mM sodium phosphate, 40 mM EDTA, 10 mM sodium sulfate, and 3-5 M urea;
(6) washing the flushed column with a buffer comprising 50-100 mM sodium phosphate, 5 mM EDTA, 10-25 mM sodium sulfate, and 2.22 mM dextrose;
(7) washing the column again with an elution buffer comprising 50-100 mM sodium phosphate, 5 mM EDTA, 150-250 mM sodium sulfate, and 0.5-1 M L-arginine;
(8) loading the resultant eluate onto a hydrophobic interaction chromatography column pre-equilibrated with 50-100 mM sodium phosphate, 150-250 mM sodium sulfate, 5 mM EDTA, and 1 M arginine;
(9) washing the resulting column with a solution comprising 100-250 mM sodium phosphate, 5 mM EDTA, and 0.8-1 M arginine; and
(10) washing the column again with a solution comprising 50-100 mM sodium phosphate, 5 mM EDTA, and 0.1-0.3 M arginine to elute the protein.

34. The method of claim 33 further comprising the steps of:
(11) concentrating the resultant eluate;
(12) filtering the retentate obtained together with a solution comprising 50 mM sodium phosphate, 0.5 M arginine;
(13) concentrating the filtered retentate; and
(14) filtering the concentrated retentate.

35. The method of claim 33, wherein said fermenting in step (1) comprises the steps of:
(a) culturing E. coli cells containing a vector comprising SEQ ID NO:8 to exponential growth phase with 2.5 to 4.5 OD₆₀₀ units in a chemically defined seed medium;
(b) inoculating cells of step (a) to a seed medium and culturing the cells to an exponential growth phase with 3.0 to 5.0 OD₆₀₀ units;
(c) transferring the cells of step (b) to a chemically defined batch medium;
(d) culturing the cells of step (c) to 25-35 units OD₆₀₀, and adding additional chemically defined medium with a feeding rate of 0.7 g/kg broth/minute;
(e) culturing the cells of step (d) to 135 to 165 units OD₆₀₀; and
(f) culturing the cells of step (e) for about four hours.

36. The method of claim 33, wherein said step (3) further comprising adding polyethyleneimine to the diluted cell culture.

37. The method of claim 33, wherein said step (10) further comprising the steps of:
(a) passing the eluate through a charged endotoxin binding filter; and
(b) flushing the filter of step (a) first with water, and then with a buffer comprising 50-100 mM sodium phosphate, 5 mM EDTA, 0.1-0.3 M arginine to elute the protein.

38. The method of claim 33, wherein said lysing in step (4) comprises passing through a high pressure homgenizer.

39. The method of claim 33, wherein said step (10) further comprising the steps of:
(a) loading the eluate onto a pre-equilibrated hydrophobic interaction chromatography column, wherein said column is equilibrated with 50-100 mM sodium phosphate, 10-100 mM ammonium sulfate, 800-1000 mM sodium chloride, 0.5-1 M arginine; and
(b) washing the column of step (a) with 50-100 mM sodium phosphate, 0.5-1 M arginine to produce an eluate.

40. The method of claim 33, wherein said step (10) further comprising the step of adding 1% polysorbate 80 or polysobate 20 to the eluate of step (10) to a final concentration of 0.01% (w/v).

41. The method of claim 33, wherein said step (10) further comprising the steps of:
(a) passing the eluate through a charged endotoxin binding filter;
(b) flushing the filter of step (a) first with water, and then with a buffer comprising 50-100 mM sodium phosphate, 5 mM EDTA, 0.1-0.3 M arginine to produce a filtrate;
(c) loading the filtrate of step (b) onto a pre-equilibrated hydrophobic interaction chromatography column, wherein said column is equilibrated with 50-100 mM sodium phosphate, 10-100 mM ammonium sulfate, 800-1000 mM sodium chloride, 0.5-1 M arginine;
(d) washing the column of step (c) with 50-100 mM sodium phosphate, 0.5-1 M arginine to produce an eluate; and
(e) adding 1% polysorbate 80 or polysorbate 20 to the eluate of step (d) to a final concentration of 0.01% (w/v).

42. The method of claim 34 further comprising a step of lyophilizing or spray drying the filtered solution of step (14).

43. One or more isolated protein produced by the method of any of claims 3242.

44. The isolated protein of claim 43 is at least 98% pure.

45. A pharmaceutical composition comprising the isolated protein of claim 43, and a pharmaceutically acceptable carrier.

46. A pharmaceutical composition comprising the isolated protein of claim 44, and a pharmaceutically acceptable carrier.

47. A formulation comprising about 0.01-1 M arginine in a salt form, sulfobutyl ether Beta-cyclodextrin sodium or sucrose, or a combination thereof, about 0.01-0.1 M sodium phosphate monobasic (NaH₂PO₄.H₂O), about 0.01%-0.1% weight/volume (“w/v”) polysorbate 80 or polysorbate 20, and the isolated protein of claim 42.

48. The formulation of claim 2 or 47 for prevention or treatment of alimentary mucositis.

49. The formulation of claim 2 or 47 for prevention or treatment of inflammatory bowel disease (“IBD”).

50. The formulation of claim 2 or 47 for prevention or treatment of osteoarthritis.

51. The formulation of claim 2 or 47 for prevention or treatment of a disorder associated with radiation exposure or a symptom thereof.

52. The formulation of claim 2 or 47 for prevention or treatment of a disorder of central nerve system.

53. The formulation of claim 2 or 47 for prevention or treatment of a cardiovascular disease.


SEQUENCE LISTING (41 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact